(19) United States
(12) Patent Application Publication
(10) Pub. No.: US 2009/0048276 A1
(43) Pub. Date: Feb. 19, 2009

(54) Inhibitors of Fatty Acid Synthase (Fas)

(76) Inventors: Mark T. Goulet, Lexington, MA (US); Benito Munoz, Brookline, MA (US); Alexey A. Rivkin, Boston, MA (US)

Correspondence Address: MERCK AND CO., INC, P O BOX 2000, RAHWAY, NJ, 07065-0907, US

(21) Appl. No.: 12/223,305

(22) PCT Filed: Jan. 26, 2007
(86) PCT No.: PCT/US2007/002254

§ 371 Date: Jul. 28, 2008

Related U.S. Application Data
(60) Provisional application No. 60/763,263, filed on Jan. 30, 2006.

(51) Int. Cl. A61K 31/519; C07D 215/20; C07D 471/04; C07D 491/04; C07D 471/12; C07D 471/14; A61P 3/06; A61P 35/00; A61K 31/4412; C07D 213/69; A61K 31/47; A61K 31/438; A61K 31/436

(52) U.S. Cl. 514/264.1; 546/157; 546/119; 546/92; 546/86; 546/116; 544/279; 544/251; 546/296; 514/312; 514/300; 514/302; 514/267; 514/348

(57) ABSTRACT

The instant invention provides for compounds which comprise substituted 3-aryl-4-hydroxyquinolin-2(1H)-ones that inhibit FAS activity. The invention also provides for compositions comprising such inhibitory compounds and methods of inhibiting FAS activity by administering the compound to a patient in need of treatment of cancer.

### BACKGROUND OF THE INVENTION

The high level of fat in the Western diet has been implicated in the development of many human malignancies including colon, breast and ovarian carcinoma. Boyd, N. F. et al. Lipids 27: 821 (1992); Risch, H. A. et al. J. Natl. Cancer Inst. 86: 1409 (1994); Risch, H. A. et al. Am. J. Epidemiol 144: 363 (1996); Carroll, K. K. Lipids 27: 793 (1996); and Reddy, B. S. Lipids 27: 807 (1992). Interestingly, data has also shown that breast cancer cells exibit high levels of fatty acid synthase (FAS) expression and activity (Kuhajda, F. P et al. Proc. Natl. Acad. Sci. USA 91: 6379 (1994), suggesting that endogenous sources of fatty acid synthesized by cancer cells promote or potentiate cancer progression.

FAS is downregulated in most normal human tissues because of the fat in our diet, with the exception of lactating breast and cycling endometrium. In contrast, FAS is often highly expressed in human cancers (breast, prostate, colon, ovary, endometrium and thyroid). This differential tissue distribution makes FAS an attractive target for cancer cells.

It is an object of the instant invention to provide novel compounds that are inhibitors of FAS.

It is also an object of the present invention to provide pharmaceutical compositions that comprise the novel compounds that are inhibitors of FAS.

It is also an object of the present invention to provide a method for treating cancer that comprises administering such inhibitors of FAS activity.

### SUMMARY OF THE INVENTION

The instant invention provides for compounds which comprise substituted 3-aryl4-hydroxyquinolin-2(1H)-ones that inhibit FAS activity. The invention also provides for compositions comprising such inhibitory compounds and methods of inhibiting FAS activity by administering the compound to a patient in need of treatment of cancer.

### DETAILED DESCRIPTION OF THE INVENTION

The compounds of the instant invention are useful in the inhibition of the activity of FAS. In a first embodiment of this invention, the inhibitors of FAS activity are illustrated by the Formula A:

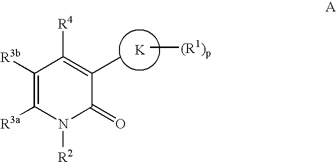

wherein:

a is 0 or 1; b is 0 or 1; m is 0, 1, or 2; p is 0, 1, 2, 3, 4 or 5;

Ring K is: aryl, C₃-C₈cycloalkyl or heterocyclyl;

R¹ is selected from: (C═O)ₐO_{b}C₁-C₁₀ alkyl, (C═O)ₐO_{b} aryl, (C═O)ₐO_{b}C₂-C₁₀ alkenyl, (C═O)ₐO_{b}C₂-C₁₀ allcnyl, CO₂H, halo, OH, O_{b}C₁-C₆ perfluoroalkyl, (C═O)ₐNR⁷R⁸, CN, (C═O)ₐO_{b}C₃-C₈ cycloalkyl, S(O)ₘNR⁷R⁸, S(O)ₘ—(C₁-C₁₀)alkyl, and (C═O)ₐO_{b}heterocyclyl, said alkyl, aryl, alkenyl, alkynyl, cycloalkyl, and heterocyclyl is optionally substituted with one or more substituents selected from R⁶;

R² is selected from: H, (C═O)ₐO_{b}C₁-C₁₀ alkyl, (C═O)ₐO_{b} aryl, (C═O)ₐO_{b}C₂-C₁₀ alkenyl, (C═O)ₐO_{b}C₂-C₁₀ alkynyl, CO₂H, halo, OH, O_{b}C₁-C₆ perfluoroalkyl, (C═O)ₐNR⁷R⁸, CN, (C═O)ₐO_{b}C₃-C₈ cycloalkyl, S(O)ₘNR⁷R⁸, S(O)ₘ—(C₁-C₁₀)alkyl, and (C═O)ₐO_{b}heterocyclyl, said alkyl, aryl, alkenyl, alkynyl, cycloalkyl, and heterocyclyl is optionally substituted with one or more substituents selected from R⁶;

R^{3a} and R^{3b} are independently selected from: H, (C═O)ₐO_{b}C₁-C₁₀ alkyl, (C═O)ₐO_{b} aryl, (C═O)ₐO_{b}C₂-C₁₀ alkenyl, (C═O)ₐO_{b}C₂-C₁₀ alkynyl, CO₂H, halo, OH, O_{b}C₁-C₆ perfluoroalkyl, (C═O)ₐNP-⁷R⁸, CN, (C═O)ₐO_{b}C₃-C₈ cycloalkyl, S(o)ₘNR⁷R⁸, S(O)ₘ—(C₁-C₁₀)alkyl, SH, NO₂, and (C═O)ₐO_{b}heterocyclyl, said alkyl, aryl, alkenyl, alkynyl, cycloalkyl, and heterocyclyl is optionally substituted with one or more substituents selected from R⁶, or R^{3a} and R^{3b} can be combined to form a fused aryl or heterocycle which are optionally substituted with one or more substituents selected from R⁶;

R⁴ is selected from: H, (C═O)ₐO_{b}C₁-C₁₀ alkyl, (C═O)ₐO_{b} aryl, (C═O)ₐO_{b}C₂-C₁₀ alkenyl, (C═O)ₐO_{b}C₂-C₁₀ alkynyl, CO₂H, halo, OH, O_{b}C₁-C₆ perfluoroalkyl, (C═O)ₐNR⁷R⁸, CN, (C═O)ₐO_{b}C₃-C₈ cycloalkyl, S(O)ₘNR⁷R⁸, S(O)ₘ—(C₁-C₁₀)alkyl, and (C═O)ₐO_{b}heterocyclyl, said alkyl, aryl, alkenyl, alkynyl, cycloalkyl, and heterocyclyl is optionally substituted with one or more substituents selected from R⁶;

R⁶ is: (C═O)ₐO_{b}C₁-C₁₀ alkyl, (C═O)ₐO_{b}aryl, C₂-C₁₀ alkenyl, C₂-C₁₀ alkynyl, (C═O)ₐO_{b} heterocyclyl, CO₂H, halo, CN, OH, O_{b}C₁-C₆ perfluoroalkyl, Oₐ(C═O)_{b}NR⁷R⁸, oxo, CHO, (N═O)R⁷R⁸, S(O)ₘNR⁷R⁸, S(O)ₘ—(C₁-C₁₀)alkyl, SH, SO₂-CF₃, NO₂, or (C═O)ₐO_{b}C₃-C₈ cycloalkyl, said alkyl, aryl, alkenyl, alkynyl, heterocyclyl, and cycloalkyl optionally substituted with one or more substituents selected from R^{6a}, and two R⁶ substituents can be combined to form a fused aryl or heterocycle which are optionally substituted with one or more substituents selected from R^{6a};

R^{6a} is selected from: (C═O)ₐO_{b}(C₁-C₁₀)alkyl, Oₐ(C₁-C₃)perfluoroalkyl, (C₀-C₆)alkylene-S(O)ₘR^{a}, oxo, OH, halo, CN, (C₂-C₁₀)alkenyl, (C₂-C₁₀)alkynyl, (C₃-C₆)cycloalkyl, (C₀-C₆)alkylene-aryl, (C₀-C₆)alkylene-heterocyclyl, (C₀-C₆)alkylene-N(R^{b})₂, C(O)R^{a}, (C₀-C₆)alkylene-CO₂R^{a}, C(O)H, and (C₀-C₆)alkylene-CO₂H, said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂;

R⁷ and R⁸ are independently selected from: H, (C═O)O_{b}C₁-C₁₀ alkyl, (C═O)O_{b}C₃-C₈ cycloalkyl, (C═O)Obaryl, (C═O)O_{b}heterocyclyl, C₁-C₁₀ alky, aryl, C₂-C₁₀ alkenyl, C₂-C₁₀ alkynyl, heterocyclyl, C₃-C₈ cycloalkyl, S(O)ₘR^{a}, and (C═O)NR^{b}₂, said alkyl, cycloalkyl, aryl, heterocylyl, alkenyl, and alkynyl is optionally substituted with one or more substituents selected from R^{6a}, or R⁷ and R⁸ can be taken together with the nitrogen to which they are attached to form a monocyclic or bicyclic heterocycle with 3-7 members in each ring and optionally containing, in addition to the nitrogen, one or two additional heteroatoms selected from N, O and S, said monocylcic or bicyclic heterocycle optionally substituted with one or more substituents selected from R^{6a};

R^{a} is H, (C₁-C₆)alkyl, (C₃-C₆)cycloalkyl, aryl, or heterocyclyl; and

R^{b} is independently H, (C₁-C₆)alkyl, aryl, heterocyclyl, (C₃-C₆)cycloalkyl, (C═O)OC₁-C₆ alkyl, (C═O)C₁-C₆ alkyl or S(O)ₘR^{a};

or a pharmaceutically acceptable salt or a stereoisomer thereof.

In a second embodiment of this invention, the inhibitors of FAS activity are illustrated by the Formula B:

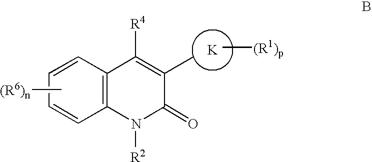

wherein:

n is 0, 1, 2, 3 or 4;

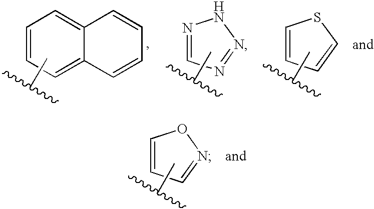

Ring K is selected from: phenyl,

all other substituents and variables are as defined in the first embodiment;

or a pharmaceutically acceptable salt or a stereoisomer thereof.

In a third embodiment of this invention, the inhibitors of FAS activity are illustrated by the Formula C:

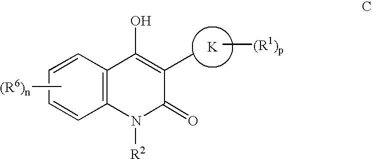

wherein:

all other substituents and variables are as defined in the second embodiment;

or a pharmaceutically acceptable salt or a stereoisomer thereof.

In a fourth embodiment of this invention, the inhibitors of FAS activity are illustrated by the Formula D:

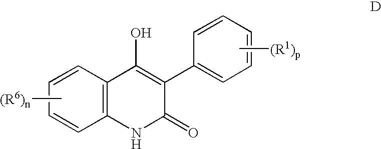

wherein:

all other substituents and variables are as defined in the second embodiment;

or a pharmaceutically acceptable salt or a stereoisomer thereof.

In a fifth embodiment of this invention, the inhibitors of FAS activity are illustrated by the Formula E:

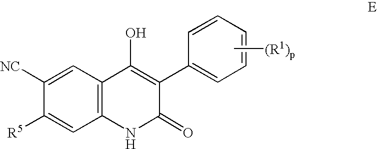

wherein:

R⁵ is selected from: H, halo and (C₁-C₆)alkyl;

all other substituents and variables are as defined in the first embodiment;

or a pharmaceutically acceptable salt or a stereoisomer thereof.

Specific compounds of the instant invention include:

7-chloro4-hydroxy-2-oxo-3-phenyl-1,2-dihydroquinoline-6-carbonitrile (1-2);

7-chloro4-hydroxy-6-nitro-3-phenylquinolin-2(1H)-one (1-3);

4-hydroxy-2-oxo-3-phenyl-1,2-dihydroquinoline-6-carbonitrile (14);

8-chloro4-hydroxy-6-nitro-3-phenylquinolin-2(1H)-one (1-5);

4-hydroxy-8-methoxy-6-nitro-3-phenylquinolin-2(1H)-one (1-6);

6,7-difluoro4-hydroxy-3-phenylquinolin-2(H1)-one (1-7);

4-hydroxy-3-phenylquinolin-2(1H)-one (1-8);

4-hydroxy-6-nitro-3-phenylquinolin-2(1H)-one (1-9);

ethyl 4-hydroxy-2-oxo-3-phenyl-1,2-dihydroquinoline-6-carboxylate (1-10);

4-hydroxy-3-phenyl-6-(trifluoromethyl)quinolin-2(1H)-one (1-11);

4-hydroxy-6-nitro-3-phenylbenzo [H]quinolin-2(1H)-one (1-12);

4-hydroxy-6-(methylsulfonyl)-3-phenylquinolin-2(1H)-one (1-13);

4-hydroxy-3-phenyl-6-[(trifluoromethyl)sulfonyl]quinolin-2(1H)-one (1-14);

4-hydroxy-7-methyl-6-nitro-3-phenylquinolin-2(1H)-one (1-15);

4-hydroxy-8-methyl-6-nitro-3-phenylquinolin-2(1H)-one (1-16);

1-ethyl4-hydroxy-6-nitro-3-phenylquinolin-2(1H)-one (1-17);

1-butyl-4-hydroxy-6-nitro-3-phenylquinolin-2(1H)-one (1-18);

methyl 7-chloro4-hydroxy-2-oxo-3-phenyl-1,2-dihydroquinoline-6-carboxylate (1-19);

4-hydroxy-1,3-dimethyl-5-phenyl-1,7-dihydro-6H-pyrazolo[3,4-b]pyridin-6-one (1-20);

1-hydroxy-2-phenyl4H-chromeno[3,4-b]pyridine-3,5-dione (1-21);

4-hydroxy-3-phenyl-1,9-dihydro-2H-pyrido[2,3-b]indol-2-one (1-22);

4-hydroxy-3-methyl-5-phenylisoxazolo[5,4-b]pyridin-6(7H)-one (1-23);

4-hydroxy-3-phenyl-7,8-dihydroquinoline-2,5(1H,6H)-dione (1-24);

4-hydroxy-3-methyl-1,5-diphenyl-1,7-dihydro-6H-pyrazolo[3,4-b]pyridin-6-one (1-25);

methyl 4-hydroxy-6-oxo-5-phenyl-6,7-dihydrofuro[2,3-b]pyridine-2-carboxylate (1-26);

5-hydroxy-1,3-dimethyl-6-phenylpyrido[2,3-d]pyrimidine-2,4,7(1H,3H,8 H)-trione (1-27);

4-hydroxy-3-phenylbenzo[b ]-1,8-naphthyridine-2,5(1H,10H)-dione (1-28);

6-hydroxy-7-phenylpyrido[3,2-e] [1,2,4]triazolo[1,5-a]pyrirnidin-8(9H)-one (1-29);

2-(4-bromophenyl)4-hydroxy-6-oxo-5-phenyl-1,6-dihydropyridine-3-carbonitrile (1-30);

4-hydroxy-8-methyl-2-oxo-3-phenyl-1,2-dihydroquinoline-6-carbonitrile (1-31);

8-ethyl-4-hydroxy-2-oxo-3-phenyl-1,2-dihydroquinoline-6-carbonitrile (1-32);

3-biphenyl-3-yl-7-chloro4-hydroxy-2-oxo-1,2-dihydroquinoline-6-carbonitrile (2-5);

7-chloro-3-(4′-fluorobiphenyl-3-yl)4-hydroxy-2-oxo-1,2-dihydroquinoline-6-carbonitrile (2-6);

7-chloro4-hydroxy-2-oxo-3-(3-pyridin-3-ylphenyl)-1,2-dihydroquinoline-6-carbonitrile (2-7);

7-chloro-3-(3′,5′-difluorobiphenyl-3-yl)4-hydroxy-2-oxo-1,2-dihydroquinoline-6-carbonitrile (2-8);

7-chloro-3-(3′,4′-difluorobiphenyl-3-yl)4-hydroxy-2-oxo-1,2-dihydroquinoline-6-carbonitrile (2-9);

-chloro4-hydroxy-3-(2′-methoxybiphenyl-3-yl)-2-oxo-1,2-dihydroquinoline-6-carbonitrile (2-10);

7-chloro4-hydroxy-3-[4-(methylsulfonyl)phenyl]-2-oxo-1,2-dihydroquinoline-6-carbonitrile (2-11);

7-chloro4-hydroxy-3-(2-methoxyphenyl)-2-oxo-1,2-dihydroquinoline-6-carbonitrile (2-12);

7-chloro-3-(2-fluorophenyl)4-hydroxy-2-oxo-1,2-dihydroquinoline-6-carboniitrile (2-13);

3-[3-(1-benzothien-3-yl)phenyl]-7-chloro4-hydroxy-2-oxo-1,2-dihydroquinoline-6-carbonitrile (2-14);

7-chloro-3-(2′,4′-difluorobiphenyl-3-yl)4-hydroxy-2-oxo-1,2-dihydroquinoline-6-carbonitrile (2-15);

7-chloro-4-hydroxy-3-[3-(methylsulfonyl)phenyl]-2-oxo-1,2-dihydroquinoline-6-carbonitrile (2-16);

7-chloro-3-(3′,5′-dichlorobiphenyl-3-yl)4-hydroxy-2-oxo-1,2-dihydroquinoline-6-carbonitrile (2-17);

7chloro4-bydroxy-3-[4′-(methylsulfonyl)biphenyl-3-yl]-2-oxo-1,2-dihydroquinoline-6-carbonitrile (2-18);

7chloro4-hydroxy-3-[3′-(methylsulfonyl)biphenyl-3-yl]-2-oxo-1,2-dihydroquinoline-6-carbonitrile (2-19);

methyl3′-(7-chloro-6-cyano4-hydroxy-2-oxo-1,2-dihydroquinolin-3-yl)biphenyl4-carboxylate (2-20);

7-chloro-4-hydroxy-3-[3-(1-methyl-1H-pyrazol4-yl)phenyl]-2-oxo-1,2-dihydroquinoline-6-carbonitrile (2-21);

4-hydroxy-6-nitro-3-phenyl-1,8-naphtbyridin-2(1H)-one (2-22);

7-chloro-4-hydroxy-3-[3-(2-naphthyl)phenyl]-2-oxo-1,2-dihydroquinoline-6-carbonitrile (2-23);

4-hydroxy-g-nitro-3-phenylquinolin-2(1H)-one (2-24);

7-fluoro-4-bydroxy-6-nitro-3-phenylquinolin-2(1H)-one (2-25);

4-hydroxy-6-nitroquinolin-2(1H)-one (2-26);

7-chloro-3-(2,4-dichlorophenyl)4-hydroxy-6-nitroquinolin-2(H)-one (2-27);

7-chloro-3-(2,4-difluorophenyl)-4-hydroxy-6-nitroquinolin-2(H)-one (2-28);

7-chloro-3-(3,5-dichlorophenyl)-4-hydroxy-6-nitroquinolin-2(1H)-one (2-29);

7-chloro-3-(3,5-difluorophenyl)14-hydroxy-6-nitroquinoin-2(1H)-one (2-30);

7-chloro-4-hydroxy-3-(2-naphthyl)-6-nitroquinolin-2(1H)-one (2-31);

3-biphenyl-yl-7-chloro 4-hydroxy-6-nitroquinolin-2(1H)-one(2-32);

3-[4-(benzyloxy)phenyl]-7-chloro 4-hydroxy-6-nitroquinolin-2(1H)-one (2-33);

4-hydroxy-2-oxo-3-phenyl-1,2-dihydroquinoline-6-carboxylic acid (2-34);

7-chloro-4-hydroxy-6-nitro-3-(2-thienyl)quinolin-2(1H)-one (2-35);

7-chloro4-hydroxy-3-(3-methylisoxazol-5-yl)-6-nitroquinolin-2(1H)-one (2-36);

7-chloro-4-hydroxy-6-nitro-3-(1H-tetrazol-5-yl)quinolin-2(1H)-one (2-37);

3-biphenyl-3-yl-7-chloro-4-hydroxy-6-nitroquinolin-2(H1)-one (2-38);

7-chloro4-[(4-methoxybenzyl)arnino]-2-oxo-3-phenyl-1,2-dihydroquinoline-6-carbonitrile (3-3);

7-chloro-2-oxo-3-phenyl-1,2-dihydroquinoline4,6-dicarbonitrile (34);

4,7-dichloro-2-oxo-3-phenyl-1,2-dihydroquinoline-6-carbonitrile (3-5);

4-(benzylamino)-7-chloro-2-oxo-3-phenyl-1,2-dihydroquinoline-6-carbonitrile (3-6); and

4-chloro-6-nitro-3-phenylquinolin-2(111)-one (3-7);

or a pharmaceutically acceptable salt or a stereoisomer thereof.

Further specific compounds of the instant invention include:

7-chloro-4-hydroxy-2-oxo-3-phenyl-1,2-dihydroquinoline-6-carbonitrile (1-2);

3-biphenyl-3-yl-7-chloro-4-hydroxy-2-oxo-1,2-dihydroquinoline-6-carbonitrile (2-5);

7-chloro-3-(4′-fluorobiphenyl-3-yl)4-hydroxy-2-oxo-1,2-dihydroquinoline-6-carbonitrile (2-6);

7-chloro-4-hydroxy-2-oxo-3-(3-pyridin-3-ylphenyl)-1,2-dihydroquinoline-6-carbonitrile (2-7);

7-chloro-3-(3′,5′-difluorobiphenyl-3-yl)4-hydroxy-2-oxo-1,2-dihydroquinoline-6-carbonitrile (2-8);

7-chloro-3-(3′,4′-difluorobiphenyl-3-yl)4-hydroxy-2-oxo-1,2-dihydroquinoline-6-carbonitrile (2-9);

-chloro-4-bydroxy-3-(2′-methoxybiphenyl-3-yl)-2-oxo-1,2-dihydroquinoline-6-carbonitrile (2-10);

7-chloro-4-hydroxy-3-[4-(methylsulfonyl)phenyl]-2-oxo-1,2-dihydroquinoline-6-carbonitrile (2-11);

7-chloro-4-hydroxy-3-(2-methoxyphenyl)-2-oxo-1,2-dihydroquinoline-6-carbonitrile (2-12);

7-chloro-3-(2-fluorophenyl)-4-hydroxy-2-oxo-1,2-dihydroquinoline-6-carbonitrile (2-13);

3-[3-(1-benzothien-3-yl)phenyl]-7-chloro-4-hydroxy-2-oxo-1,2-dihydroquinoline-6-carbonitrile (2-14);

7-chloro-3-(2¹′,4-difluorobiphenyl-3-yl)-4-hydroxy-2-oxo-1,2-dihydroquinoline-6-carbonitrile (2-15);

7-chloro-4-hydroxy-3-[3-(methylsulfonyl)phenyl]-2-oxo-1,2-dihydroquinoline-6-carbonitrile (2-16);

7-chloro-3-(3′,5′-dichlorobiphenyl-3-yl)4-hydroxy-2-oxo-1,2-dihydroquinoline-6-carbonitrile (2-17);

7-chloro4-hydroxy-3-[4′-(methylsulfonyl)biphenyl-3-yl]-2-oxo-1,2-dihydroquinoline-6-carbonitrile (2-18);

7-chloro4-hydroxy-3-[3′-(methylsulfonyl)biphenyl-3-yl]-2-oxo-1,2-dihydroquinoline-6-carbonitrile (2-19);

methyl3′-(7-chloro-6-cyano4-hydroxy-2-oxo-1,2-dihydroquinolin-3-yl)biphenyl-4-carboxylate (2-20);

7-chloro4-hydroxy-3-[3-(1-methyl-1H-pyrazol4-yl)phenyl]-2-oxo-1 ,2-dihydroquinoline-6-carbomitrile (2-21); and

7-chloro4-hydroxy-3-[3-(2-naphthyl)phenyl]-2-oxo-1,2-dihydroquinoline-6-carbonitrile (2-23);

or a pharmaceutically acceptable salt or a stereoisomer thereof.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, being included in the present invention. In addition, the compounds disclosed herein may exist as tautomers and both tautomeric forms are intended to be encompassed by the scope of the invention, even though only one tautomeric structure is depicted.

When any variable (e.g. R¹, R⁶, R^{6a}, etc.) occurs more than one time in any constituent, its definition on each occurrence is independent at every other occurrence. Also, combinations of substituents and variables are permissible only if such combinations result in stable compounds. Lines drawn into the ring systems from substituents indicate that the indicated bond may be attached to any of the substitutable ring atoms. If the ring system is bicyclic, it is intended that the bond be attached to any of the suitable atoms on either ring of the bicyclic moiety.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases the preferred embodiment will have from zero to three substituents.

It is understood that one or more Si atoms can be incorporated into the compounds of the instant invention by one of ordinary skill in the art to provide compounds that are chemically stable and that can be readily synthesized by techniques known in the art from readily available starting materials.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on.

The term “cycloalkyl” means a monocyclic or bicyclic saturated aliphatic hydrocarbon group having the specified number of carbon atoms. For example, “cycloalkyl” inlcudes cyclopropyl, methyl-cyclopropyl, 2,2-dimethyl-cyclobutyl, 2-ethyl-cyclopentyl, cyclohexyl, and so on.

“Alkoxy” represents either a cyclic or non-cyclic alkyl group of indicated number of carbon atoms attached through an oxygen bridge. “Alkoxy” therefore encompasses the definitions of alkyl and cycloallyl above.

If no number of carbon atoms is specified, the term “alkenyl” refers to a non-aromatic hydrocarbon radical, straight, branched or cyclic, containing from 2 to 10 carbon atoms and at least one carbon to carbon double bond. Preferably one carbon to carbon double bond is present, and up to four non-aromatic carbon-carbon double bonds may be present. Thus, “C₂-C₆ alkenyl” means an alkenyl radical having from 2 to 6 carbon atoms. Alkenyl groups include ethenyl, propenyl, butenyl, 2-methylbutenyl and cyclohexenyl. The straight, branched or cyclic portion of the alkenyl group may contain double bonds and may be substituted if a substituted alkenyl group is indicated.

The term “alkynyl” refers to a hydrocarbon radical straight, branched or cyclic, containing from 2 to 10 carbon atoms and at least one carbon to carbon triple bond. Up to three carbon-carbon triple bonds may be present. Thus, “C₂-C₆ alkynyl” means an alkynyl radical having from 2 to 6 carbon atoms. Alkynyl groups include ethynyl, propynyl, butynyl, 3-methylbutynyl and so on. The straight, branched or cyclic portion of the alcynyl group may contain triple bonds and may be substituted if a substituted alkynyl group is indicated.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term heteroaryl, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include: acridinyl, carbazolyl, cinnolinyl, quinoxalinyl, pyrrazolyl, indolyl, benzotriazolyl, furanyl, thienyl, benzothienyl, benzofuiranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-ontaining heteroaryl. In cases where the heteroaryl substituent is bicyclic and one ring is non-aromatic or contains no heteroatoms, it is understood that attachment is via the aromatic ring or via the heteroatom containing ring, respectively.

As appreciated by those of skill in the art, “halo” or “halogen” as used herein is intended to include chloro (Cl), fluoro (F), bromo (Br) and iodo (I).

The term “heterocycle” or “heterocyclyl” as used herein is intended to mean a 3- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include: benzoimidazolyl, benzofuranyl, benzofurazanyl, benzopyrazolyl, benzotriazolyl, benzothiophenyl, benzoxazolyl, carbazolyl, carbolinyl, chromanyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisochromenyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydroisoquinolinyl, tetrahydroquinolinyl and tetrahydrothienyl, and N-oxides thereof. Attachment of a heterocyclyl substituent can occur via a carbon atom or via a heteroatom.

The alkyl, alkenyl, alkynyl, cycloalkyl, aryl, heteroaryl and heterocyclyl substituents may be unsubstituted or unsubstituted, unless specifically defined otherwise. For example, a (C₁-C₆)alkyl may be substituted with one, two or three substituents selected from OH, oxo, halogen, alkoxy, dialkylamino, or heterocyclyl, such as morphboinyl, piperidinyl, and so on. In this case, if one substituent is oxo and the other is OH, the following are included in the definition: —(C═O)CH₂CH(OH)CH₃, —(C═O)OH, —CH₂(OH)CH₂CH(O), and so on.

In certain instances, R⁷ and R⁸ are defined such that they can be taken together with the nitrogen to which they are attached to form a monocyclic or bicyclic heterocycle with 3-7 members in each ring and optionally containing, in addition to the nitrogen, one or two additional heteroatoms selected from N, O and S, said heterocycle optionally substituted with one or more substituents selected from R^{6a}. Examples of the heterocycles that can thus be formed include the following, keeping in mind that the heterocycle is optionally substituted with one or more substituents chosen from R^{6a}:

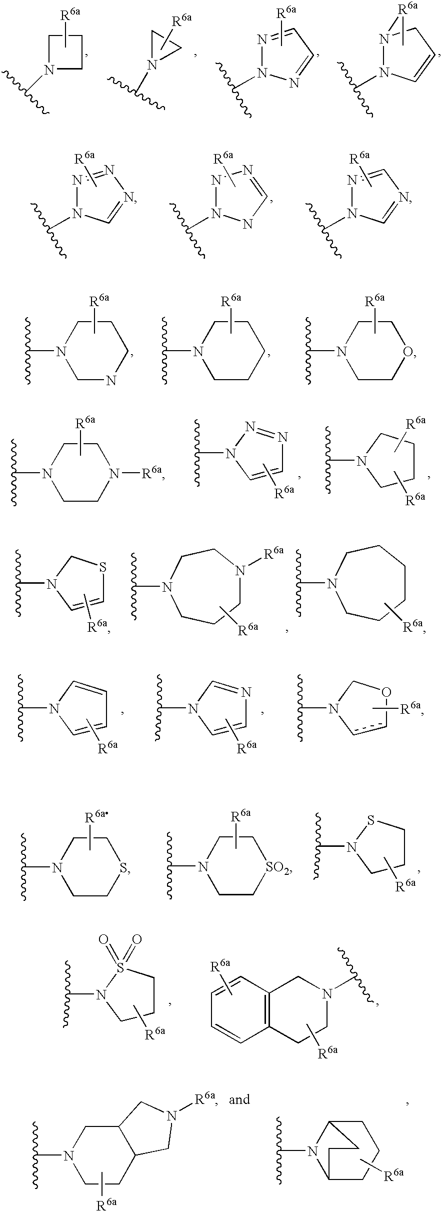

In another embodiment of Formula B, Ring K is aryl or heterocyclyl.

In another embodiment of Formula B, Ring K is aryl or heterocyclyl and R² is H or (C₁-C₆)alkyl.

In another embodiment of Formula C, n is 2, R² is H or (C₁-C₆)allkyl, and one R⁶ is CN and the other R⁶ is Cl.

In another embodiment of Formula E, R¹ is selected from: H, halo, (C₁-C₆)alkyl, aryl, heterocyclyl, O(C₁-C₆)alkyl, S(O)₂—(C₁-C₆)alkyl and phenyl, wherein said allyl, phenyl, aryl and heterocylcyl are optionally substituted with from one to five substituents selected from, H, halo, phenyl, O(C₁-C₆)alkyl, (C₁-C₆)alkyl, (C═O)O(C₁-C₆)alkyl, S(O)₂—(C₁-C₆)alkyl, OH, oxo, CF₃ and NH₂, and R⁵ is Cl.

In another embodiment of Formula E, R¹ is selected from: H and phenyl, wherein said phenyl is optionally substituted with from one to five substituents selected from, H, halo, phenyl, O(C₁-C₆)alkyl, (C₁-C₆)alkyl, (C═O)O(C₁-C₆)alkyl, S(O)₂—(C₁-C₆)alkyl, OH, oxo, CF₃ and NH₂, and R⁵ is Cl.

Included in the instant invention is the free form of compounds of Formula A, as well as the pharmaceutically acceptable salts and stereoisomers thereof. Some of the isolated specific compounds exemplified herein are the protonated salts of amine compounds. The term “free form” refers to the amine compounds in non-salt form. The encompassed pharmaceutically acceptable salts not only include the isolated salts exemplified for the specific compounds described herein, but also all the typical pharmaceutically acceptable salts of the free form of compounds of Formula A. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic (TFA) and the like.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammnonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucarnine, morpholine, piperazine, piperidine, polyamine resins, procaine, purines, theobromine, triethylamine, trimethylamine tripropylamine, tromethamine and the like.

The preparation of the pharmaceutically acceptable salts described above and other typical pharmaceutically acceptable salts is more fully described by Berg el al., “Pharmaceutical Salts,” J. Pharm. Sci., 1977:66:1-19.

It will also be noted that the compounds of the present invention are potentially internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom.

### Utility

The compounds, compositions and methods provided herein are particularly deemed useful for the treatment of cancer. Cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deforrnans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

Cancers that may be treated by the compounds, compositions and methods of the invention include, but are not limited to: breast, prostate, colon, colorectal, lung, brain, testicular, stomach, pancrease, skin, small intestine, large intestine, throat, head and neck, oral, bone, liver, bladder, kidney, thyroid and blood.

Cancers that may be treated by the compounds, compositions and methods of the invention include breast, prostate, colon, ovary, endometrium and thyroid.

Cancers that may be treated by the compounds, compositions and methods of the invention include breast and prostate.

The compounds of the invention are also useful in preparing a medicament that is useful in treating cancer.

The compounds of this invention may be administered to mammals, including humans, either alone or, in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, cellulose acetate buryrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxyrnethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an allylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavouring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula A may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula A are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

When a composition according to this invention is administered into a human subject, the daily dosage will normally be determined by the prescribing physician with the dosage generally varying according to the age, weight, and response of the individual patient, as well as the severity of the patient's symptoms.

In an embodiment, a suitable amount of an inhibitor of FAS is administered to a mammal undergoing treatment for cancer. Administration occurs in an amount of inhibitor of between about 0.1 mg/kg of body weight to about 60 mg/kg of body weight per day, or between 0.5 mg/kg of body weight to about 40 mg/kg of body weight per day. Another therapeutic dosage that comprises the instant composition includes from about 0.01 mg to about 1000 mg of inhibitor of FAS. In another embodiment, the dosage comprises from about 1 mg to about 1000 mg of inhibitor of FAS.

The instant compounds are also useful in combination with therapeutic, chemotherapeutic and anti-cancer agents. Combinations of the presently disclosed compounds with therapeutic, chemotherapeutic and anti-cancer agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, -y-secretase inhibitors, agents that interfere with receptor tyrosine kinases (RTKs) and agents that interfere with cell cycle checkpoints. The instant compounds are particularly useful when co-administered with radiation therapy.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy4-rmethyl-2-[4-[2-(l -piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′deamino-3′-morpholino-13-deoxo-10-hydroxycarminomycin, annamycin, galarubicin, elinafide, MEN10755, 4-demethoxy-3-deamino-3-aziridinyl-4-methylsulphoxnyl-daunorubicin (see WO 00/50032), Raf kinase inhibitors (such as Bay43-9006) and rnTOR inhibitors (such as Wyeth's CCI-779).

An example of a hypoxia activatable compound is tirapazamine.

Examples of proteosome inhibitors include but are not limited to lactacystin and MLN-341 (Velcade).

Examples of microtubule inhibitors/microtubule-stabilising agents include paclitaxel, vindesine sulfate, 3′,4′-didehydro4′-deoxy-8′-norvincaleukoblastine, docetaxol, rhizoxin, dolastatin, mivobulin isethionate, auristatin, cemadotin, RPRl09881, BMS184476, vinflunine, cryptophycin, 2,3,4,5,6-pentafluoro-N-(3-fluoro4-methoxyphenyl) benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS 188797. In an embodiment the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(di7ethylamino)etnyl]-N-methylamino]ethyl]-5-[4-bydro7xy-3,5-dimethoxyphenyl]-5,5a,6,8,8a,9-hexohydrofuro(3′,4′:6,7)naphtho(2,3-d)-1,3-dioxol-6-one, 2,3-(metbylenedioxy)-5-methyl-7-hydroxy-8-methoxybenzo[c]-phenanthridinium, 6,9-bis[(2-aminoethyl)amino]benzo[g] isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen4-ylmethyl]formamide, N-(2-(dimethylamino)ethyl)acridine4-carboxamide, 6-[[2-(dimethylamino)ethyl]amino]-3-hydroxy-7H-indeno[2,1-c] quinolin-7-one, and dimesna.

Examples of inhibitors of mitotic kinesins, and in particular the human mitotic kinesin KSP, are described in PCT Publications WO 01/30768 and WO 01/98278, and pending U.S. Ser. Nos. 60/338,779 (filed Dec. 6, 2001), 60/338,344 (filed Dec. 6, 2001), 60/338,383 (filed Dec. 6, 2001), 60/338,380 (filed Dec. 6, 2001), 60/338,379 (filed Dec. 6, 2001) and 60/344,453 (filed Nov. 7, 2001). In an embodiment inhibitors of mitotic kinesins include, but are not limited to inhibitors of KSP, inhibitors of MKLP1, inhibitors of CENP-E, inhibitors of MCAK and inhibitors of Rab6-KIFL

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy4-[N2-[2(E),4(E)-tetradecadienoyl]glycylamino]-L-glycero-B-L-manno-heptopyranosyl]adenine, aplidine, ecteinascidin, troxacitabine, 4-[2-amino4-oxo4,6,7,8-tetrahydro-3H-pyrimidino[5,4-b] [1,4]thiazin-6-yl-(S)ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No.5,177,080). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95132987, U.S. Pat. No. 5,420,245, U.S. Pat. No. 5,523,430, U.S. Pat. No. 5,532,359, U.S. Pat. No.5,510,510, U.S. Pat. No.5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No.5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No.5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97144350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFRI) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflarmnatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in U.S. Ser. Nos. 60/310,927 (filed Aug. 8, 2001) and 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the CHK11 and CHK12 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001.

“Inhibitors of cell proliferation and survival signalling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, WO 02/083138, WO 03/086279, WO 03/086394, WO 03/086403, WO 03/086404 and WO 04/041162), inhibitors of Raf kinase (for example BAY-43-9006), inhibitors of MEK (for example CI-1040 and PD-098059), inhibitors of mTOR (for example Wyeth CCI-779), and inhibitors of P13K (for example LY294002).

As described above, the combinations with NSAID's are directed to the use of NSAID's which are potent COX-2 inhibiting agents. For purposes of this specification an NSAID is potent if it possesses an IC₅₀ for the inhibition of COX-2 of 1 μM or less as measured by cell or microsomal assays.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272 and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine; or a pharmaceutically acceptable salt thereof.

Compounds that have been described as specific inhibitors of COX-2 and are therefore usefuil in the present invention include, but are not limited to, the following: parecoxib, BEXTRA® and CELEBREX® or a pharmaceutically acceptable salt thereof.

Other examples of angiogenesis inhibitors include, but are not limited to, endostatin, ukrain, ranpimase, IM862, 5-methoxy-4-[2-methyl-3-(3-methyl-2-butenyl)oxiranyl]-oxaspiro[2,5]oct-6-yl(chloroacetyl)carbamate, acetyldinanaline, 5-amino-1-[[3,5-dichloro4-(4-chlorobenzoyl)phenyl]methyl]-1H-1,2,3-triazole-4-carboxamide,CM101, squalamine, combretastatin, RPI4610, NX31838, sulfated mannopentaose phosphate, 7,7-(carbonyl-bis[imino-N-methyl4,2-pyrrolocarbonylimino[N-methyl4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αvβ₃ integrin, to compounds which selectively antagonize, inhibit or counteract binding of a physiological ligand to the αvβ5 integrin, to compounds which antagonize, inhibit or counteract binding of a physiological ligand to both the αvβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382,2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563,4-(3-chlorophenylamino)-5,6-dimetbyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU666 8, STI571A, N4-chl orophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Phartnacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW233 1, GW409544, NN2344, KRP297, NP0110, DRF4158, NN622, GI262570, PNU182716, DRF552926,2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro4-(4-fluorophenoxy) phenoxy)propoxy)-2-ethylchromane-2-carboxylic acid (disclosed in U.S. Ser. Nos. 60/235,708 and 60/244,697).

Another embodiment of the instant invention is the use of the presently disclosed compounds in combination with gene therapy for the treatment of cancer. For an overview of genetic strategies to treating cancer see Hall et al (Am. J. Hum. Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8):1105-13), and interferon gamma (J. Immunol. 2000;164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another embodiment, conjunctive therapy with an anti-emesis agent selected from a neurokinin-1 receptor antagonist, a 5HT3 receptor antagonist and a corticosteroid is disclosed for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989,0 428 434, 0 429 366, 0 430 771, 0 436 334, 0 443 132, 0 482 539, 0 498 069, 0 499 313, 0 512 901, 0 512 902,0 514 273,0 514 274,0 514 275,0 514 276,0 515 681, 0 517 589, 0 520 555, 0 522 808, 0 528 495, 0 532 456, 0 533 280, 0 536 817, 0 545 478, 0 558 156, 0 577 394, 0 585 913,0 590 152, 0 599 538, 0 610 793, 0 634 402, 0 686 629, 0 693 489, 0 694 535, 0 699 655, 0 699 674, 0 707 006,0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042,95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418,95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)phenyl)etboxy)-3-(S)-(4-fluorophenyl)4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be administered with an agent useful in the treatment of anemia. Such an anemia treatment agent is, for example, a continuous erythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levarnisole, isoprinosine and Zadaxin.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be useful for treating or preventing breast cancer in combination with aromatase inhibitors. Examples of aromatase inhibitors include but are not limited to: anastrozole, letrozole and exemestane.

A compound of the instant invention may also be useful for treating or preventing cancer in combination with siRNA therapeutics.

A compound of the instant invention may also be useful for treating or preventing cancer in combination with γ-secretase inhibitors.

Thus, the scope of the instant invention encompasses the use of the instantly claimed compounds in combination with a second compound selected from: an estrogen receptor modulator, an androgen receptor modulator, a retinoid receptor modulator, a cytotoxic/cytostatic agent, an antiproliferative agent, a prenyl-protein transferase inhibitor, an H-MG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, γ-secretase inhibitors; agents that interfere with receptor tyrosine kinases (RTKs) and an agent that interferes with a cell cycle checkpoint.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In an embodiment, the angiogenesis inhibitor to be used as the second compound is elected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-l, or an antibody to VEGF. In an embodiment, the estrogen receptor modulator is tamoxifen or raloxifene.

Also included in the scope of the claims is a method of treating cancer that comprises administering a therapeutically effective amount of a compound of the instant invention in combination with radiation therapy and/or in combination with a second compound selected from: an estrogen receptor modulator, an androgen receptor modulator, a retinoid receptor modulator, a cytotoxiceytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, PPAR-γ agonists, PPAR-δ agonists, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, a bisphosphonate, an aromatase inhibitor, an siRNA therapeutic, -y-secretase inhibitors, agents that interfere with receptor tyrosine kinases (RTKs) and an agent that interferes with a cell cycle checkpoint.

And yet another embodiment of the invention is a method of treating cancer that comprises administering a therapeutically effective amount of a compound of the instant invention in combination with paclitaxel or trastuzumab.

The invention further encompasses a method of treating or preventing cancer that comprises administering a therapeutically effective amount of a compound of the instant invention in combination with a COX-2 inhibitor.

The instant invention also includes a pharmaceutical composition useful for treating or preventing cancer that comprises a therapeutically effective amount of a compound of the instant invention and a second compound selected from: an estrogen receptor modulator, an androgen receptor modulator, a retinoid receptor modulator, a cytotoxiclcytostatic agent, an antiproliferative agent, a prenyl-protein transferase inhibitor, an HIG-CoA reductase inhibitor, an HIV protease inhibitor, a reverse transcriptase inhibitor, an angiogenesis inhibitor, a PPAR-γ agonist, a PPAR-δ agonist, an inhibitor of cell proliferation and survival signaling, a bisphosphonate, an aromatase inhibitor, an siRNA therapeutic, γ-secretase inhibitors, agents that interfere with receptor tyrosine kinases (RTKs) and an agent that interferes with a cell cycle checkpoint.

All patents, publications and pending patent applications identified are hereby incorporated by reference.

Abbreviations used in the description of the chemistry and in the Examples that follow are: Ac₂O (acetic anhydride); AcOH (acetic acid); AEBSE (p-aminoethylbenzenesulfonyl fluoride); BSA (bovine serum albumin); BuLi (n-Butyl lithium); CDCl₃ (chloroform-d); Cul (copper iodide); CuSO₄ (copper sulfate); DBU (1,8-DLAZABICYCLO[5.4.0]LJNDEC-7-ENE); DCE (dichloroethane); DCM (dichloromethane); DEAD (diethyl azodicarboxylate); DIPEA (diisopropylethylamine); DMF (N,N-dimethylformanide); DMP (Dess-Martin periodinane); DMSO (dimethyl sulfoxide); DPPA (diphenylphosphoryl azide); DTT (dithiothreitol); EDTA (ethylene-diamine-tetra-acetic acid); EGTA (ethylene-glycol-tetra-acetic acid); Et₂O (diethylether); EtOAc (ethyl acetate); EtOH (ethanol); HOAc (acetic acid); HPLC (high-performance liquid chromatography); HRMS (high resolution mass spectrum); LAH (lithium aluminum hydride); LCMS (liquid chromatograph-mass spectrometer); LHIDS (lithium bis(trimethylsilyl)amide); LRMS (low resolution mass spectrum); mCPBA (3-chloroperoxybenzoic acid); MeOH (methanol); MP-B(CN)H₃ (Macroporous cyanoborohydride); NaHCO₃ (sodium bicarbonate); Na₂SO₄ (sodium sulfate); Na(OAc)₃BH (sodium triacetoxyborohydride); NH₄OAc (arnmonium acetate); NBS (N-bromosuccinamide); NMP (1-methyl-2-pyrrolidinone); NMR (nuclear magnetic resonance); PBS (phosphate buffered saline); PCR (polymerase chain reaction); Pd(dppi) ([1,1′-bis(diphenylphosphino)ferrocene] palladium); Pd(Ph₃)₄ (palladium(0) tetrakis-triphenylphosphine); POCl₃ (phosphorous oxychloride); PS-DIEA (polystyrene diisopropylethylamine); PS-PPh₃ (polystyrene-triphenyl phosphine); PTSA (para-toluene sulfonic acid); Pyr (pyridine); Selectfluor (1-chloromethyl-4-fluoro-1,4-diazoniabicyclo[2.2.2]octane bis(tetrafluoroborate); TBAF (tetrabutylammonium fluoride); T-BuOH (tert-butanol); THF (tetrahydrofuran); Tf (trifluoromethanesulfonyl); TFA (trifluoroacteic acid); and TMSCH₂N₂ (trimethylsilyldiazomethane).

The compounds of this invention may be prepared by employing reactions as shown in the following Reaction Schemes, in addition to other standard manipulations that are known in the literature or exemplified in the experimental procedures. The illustrative Reaction Schemes below, therefore, are not limited by the compounds listed or by any particular substituents employed for illustrative purposes. Substituent numbering as shown in the Reaction Schemes do not necessarily correlate to that used in the claims and often, for clarity, a single substituent is shown attached to the compound where multiple substituents are optionally allowed under the definitions of Formula A hereinabove.

Reactions used to generate the compounds of this invention are prepared by employing reactions as shown in Reaction Schemes I-III.

### Synopsis of Reaction Schemes

As shown in Reaction Scheme I, solvent-free microwave reaction of enamines, anilines, heterocyclic anilines with di-(2,4,6-trichlorophenyl)-2-phenylmalonate provide the corresponding 3-arylpyridone (I-A) or 3-aryl4-hydroxyquinolin-2(1H)-ones (I-A).

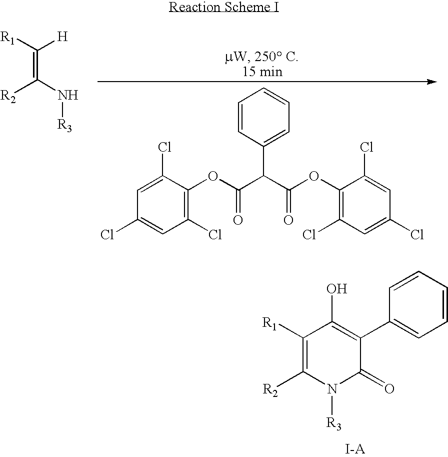

Reaction Scheme II illustrates how 4-hydroxy-3-phenylquinoline-2(1H)-one (B-4) can be prepared from cyclization of B-3, which is prepared by acylation of B-2 with B-1.

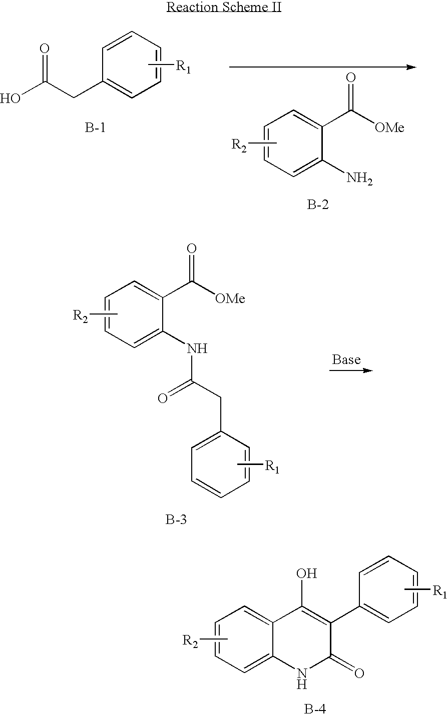

Reaction Scheme IIIillustrates how 4-substituent-3-phenylquinoline-2(1H)-one (C-3) can be prepared from C-2, which can prepared from C-1.

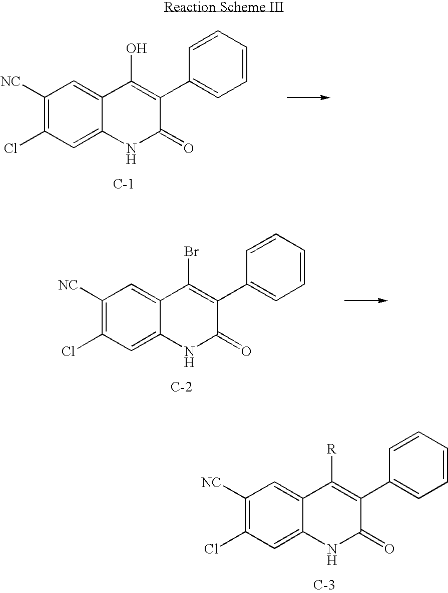

### EXAMPLES

Examples provided are intended to assist in a further understanding of the invention. Particular materials employed, species and conditions are intended to be further illustrative of the invention and not limitative of the reasonable scope thereof. The reagents utilized in synthesizing the compounds depicted in the following Tables are either commercially available or are readily prepared by one of ordinary skill in the art.

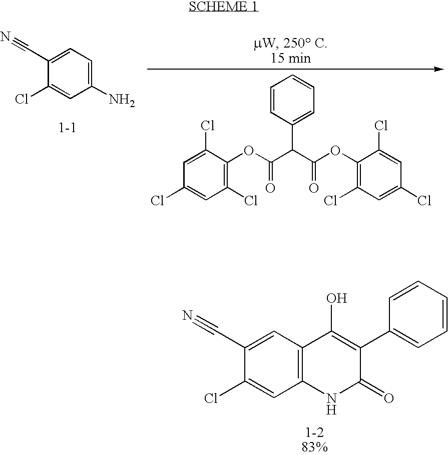

7chloro-4-hydroxy-2-oxo-3-phenyl-1,2-dihvdroquinoline-6-carbonitrile (1-2)

A stirred mixture of 4-amino-2-chlorobenzonitrile (1-1; 0.076 g) and di-(2,4,6-trichlorophenyl)-2-phenylmalonate (0.535 g) in a sealed tube was irradiated at 250° C. for 15 minutes. The crude reaction mixture was treated with diethyl ether at room temperature and the precipitate collected by vacuum filtration and dried under high vacuum for 2 hours. ¹H-NMR (600 MHz, d₆-DMSO) δ=11.9 (1H, s), 10.7 (1H, s), 8.39 (1H, s), 7.44 (1H, t, J=7.5 Hz); 7.39-7.37 (2H, m), 7.33-7.31 (3H, m); ¹³C-NMR (150 MHz, d6-DMSO) δ=163.4, 156.9, 142.2, 136.3, 133.1, 131.8, 131.7, 128.4, 127.9, 116.9, 116.3, 20 115.6, 114.7, 104.5; LC-ESMS observed [M+H]+297.0 (calcd 297.0).

The compounds shown in Table 1 were synthesized according to Reaction Scheme 1.

TABLE 1

| Cmp | Structure | Name | LRMS m/z (M + H) |
|---|---|---|---|
| 1-3 | 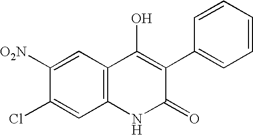 | 7-chloro-4-hydroxy-6-nitro-3-phenylquinolin-2(1H)-one | LRMS m/z (M + H)316.1 found, 316.1required. |
| 1-4 | 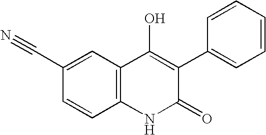 | 4-hydroxy-2-oxo-3-phenyl-1,2-dihydroquinoline-6-carbonitrile | LRMS m/z (M + H)263.1 found, 263.1required. |
| 1-5 | 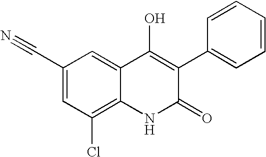 | 8-chloro-4-hydroxy-6-nitro-3-phenylquinolin-2(1H)-one | LRMS m/z (M + H)297.0 found, 297.0required. |
| 1-6 | 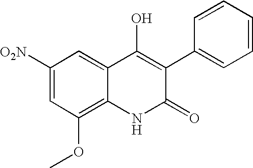 | 4-hydroxy-8-methoxy-6-nitro-3-phenylquinolin-2(1H)-one | LRMS m/z (M + H)313.1 found, 313.1required. |
| 1-7 | 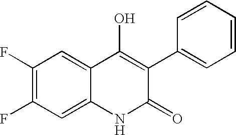 | 6,7-difluoro-4-hydroxy-3-phenylquinolin-2(1H)-one | LRMS m/z (M + H)274.1 found, 274.1required. |
| 1-8 | 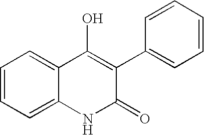 | 4-hydroxy-3-phenylquinolin-2(1H)-one | LRMS m/z (M + H)238.1 found, 238.1required. |
| 1-9 | 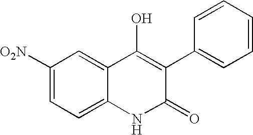 | 4-hydroxy-6-nitro-3-phenylquinolin-2(1H)-one | LRMS m/z (M + H)283.1 found, 283.1required. |
| 1-10 | 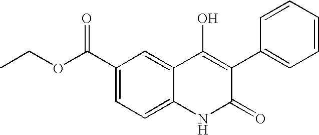 | ethyl 4-hydroxy-2-oxo-3-phenyl-1,2-dihydroquinoline-6-carboxylate | LRMS m/z (M + H)310.1 found, 310.1required. |
| 1-11 | 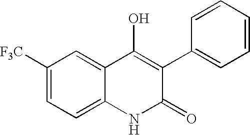 | 4-hydroxy-3-phenyl-6-(trifluoromethyl)quinolin-2(1H)-one | LRMS m/z (M + H)306.1 found, 306.1required. |
| 1-12 | 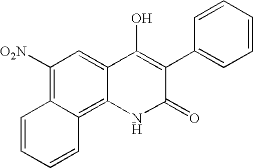 | 4-hydroxy-6-nitro-3-phenylbenzo[H]quinolin-2(1H)-one | LRMS m/z (M + H)333.1 found, 333.1required. |
| 1-13 | 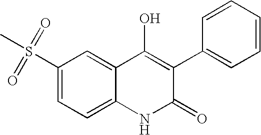 | 4-hydroxy-6-(methylsulfonyl)-3-phenylquinolin-2(1H)-one | LRMS m/z (M + H)316.1 found, 316.1required. |
| 1-14 | 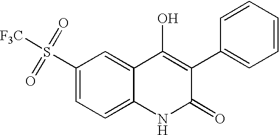 | 4-hydroxy-3-phenyl-6-[(trifluoromethyl)sulfonyl]quinolin-2(1H)-one | LRMS m/z (M + H)370.0 found, 370.0required. |
| 1-15 | 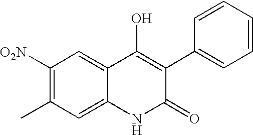 | 4-hydroxy-7-methyl-6-nitro-3-phenylquinolin-2(1H)-one | LRMS m/z (M + H)297.1 found, 297.1required. |
| 1-16 | 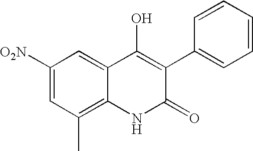 | 4-hydroxy-8-methyl-6-nitro-3-phenylquinolin-2(1H)-one | LRMS m/z (M + H)297.1 found, 297.1required. |
| 1-17 | 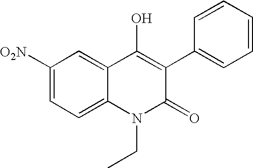 | 1-ethyl-4-hydroxy-6-nitro-3-phenylquinolin-2(1H)-one | LRMS m/z (M + H)311.1 found, 311.1required. |
| 1-18 | 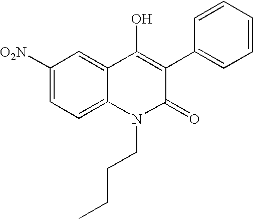 | 1-butyl-4-hydroxy-6-nitro-3-phenylquinolin-2(1H)-one | LRMS m/z (M + H)339.1 found, 339.1required. |
| 1-19 | 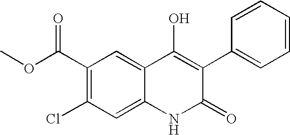 | methyl 7-chloro-4-hydroxy-2-oxo-3-phenyl-1,2-dihydroquinoline-6-carboxylate | LRMS m/z (M + H)330.0 found, 330.0required. |
| 1-20 | 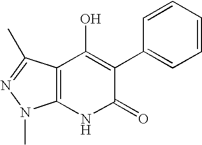 | 4-hydroxy-1,3-dimethyl-5-phenyl-1,7-dihydro-6H-pyrazolo[3,4-b]pyridin-6-one | LRMS m/z (M + H)256.1 found, 256.1required. |
| 1-21 | 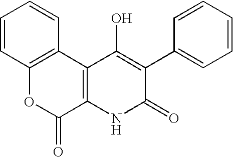 | 1-hydroxy-2-phenyl-4H-chromeno[3,4-b]pyridine-3,5-dione | LRMS m/z (M + H)306.1 found, 306.1required. |
| 1-22 | 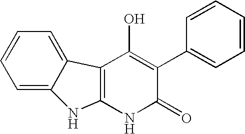 | 4-hydroxy-3-phenyl-1,9-dihydro-2H-pyrido[2,3-b]indol-2-one | LRMS m/z (M + H)277.1 found, 277.1required. |
| 1-23 | 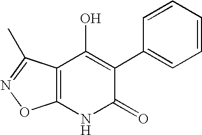 | 4-hydroxy-3-methyl-5-phenylisoxazolo[5,4-b]pyridin-6(7H)-one | LRMS m/z (M + H)243.1 found, 243.1required. |
| 1-24 | 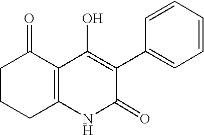 | 4-hydroxy-3-phenyl-7,8-dihydroquinoline-p2,5(1H,6H)-dione | LRMS m/z .(M + H)256.1 found, 256.1required. |
| 1-25 | 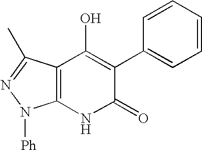 | 4-hydroxy-3-methyl-1,5-diphenyl-1,7-dihydro-6H-pyrazolo[3,4-b]pyridin-6-one | LRMS m/z (M + H)318.1 found, 318.1required. |
| 1-26 | 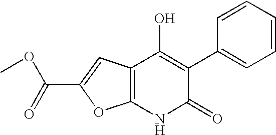 | methyl 4-hydroxy-6-oxo-5-phenyl-6,7-dihydrofuro[2,3-b]pyridine-2-carboxylate | LRMS m/z (M + H)286.1 found, 286.1required. |
| 1-27 | 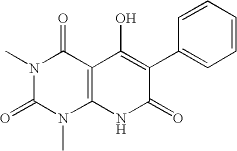 | 5-hydroxy-1,3-dimethyl-6-phenylpyrido[2,3-d]pyrimidine-2,4,7(1H,3H,8H)-trione | LRMS m/z (M + H)300.1 found, 300.1required. |
| 1-28 | 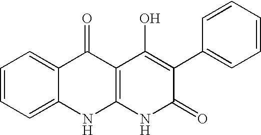 | 4-hydroxy-3-phenylbenzo[b]-1,8-naphthyridine-2,5(1H,10H)-dione | LRMS m/z (M + H)305.1 found, 305.1required. |
| 1-29 | 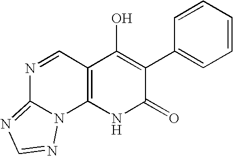 | 6-hydroxy-7-phenylpyrido[3,2-e)[1,2,4]triazolo[1,5-a]pyrimidin-8(9H)-one | LRMS m/z (M + H)280.1 found, 280.1required. |
| 1-30 | 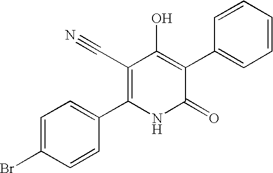 | 2-(4-bromophenyl)-4-hydroxy-6-oxo-5-phenyl-1,6-dihydropyridine-3-carbonitrile | LRMS m/z (M + H)367.0 found, 367.0required. |
| 1-31 | 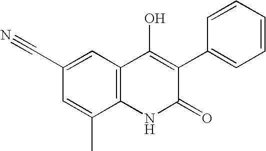 | 4-hydroxy-8-methyl-2-oxo-3-phenyl-1,2-dihydroquinoline-6-carbonitrile | LRMS m/z (M + H)277.1 found, 277.1required. |
| 1-32 | 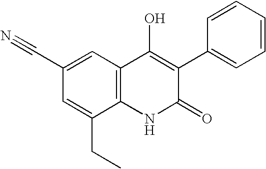 | 8-ethyl-4-hydroxy-2-oxo-3-phenyl-1,2-dihydroquinoline-6-carbonitrile | LRMS m/z (M + H)291.1 found, 291.1required. |

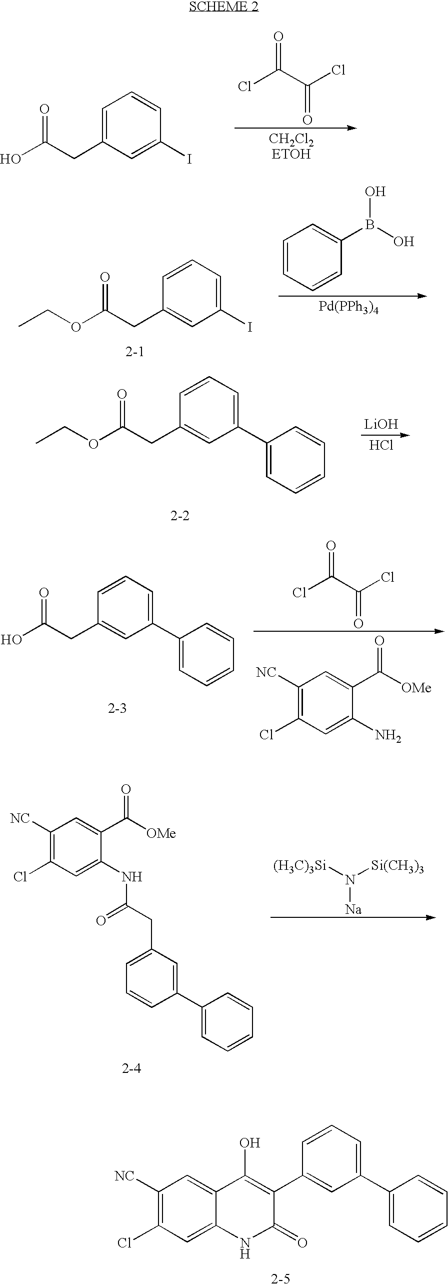

3-biphenyl-3-yl-7-chloro-4-hydroxy-2-oxo-1,2-dihydroquinoline-6-carbonitrile (2-5)

ethyl (3-iodophenyl)acetate (2-1)

To a solution of 3-iodophenylacetic acid (24.8 g, 94.6 mmol) in CH₂Cl₂ (200 mL) was added oxalyl chloride (189 mmol, 2 equiv), followed by a drop of DMF. The mixture was stirred at room temperature for lh and then concentrated. Addition of ethanol and purification by flash chromatography (EtOAc/Hex) gave yellow oil as product. ¹H NMR (600 MHz, CDCl₃): δ=7.63 (s, 1 H), 7.59 (d, 1 H), 7.25 (d, 1 H), 7.04 (t, 1 H), 4.16 (q, 2 H), 3.53 (s,2 H), 1.25 (t, 3 H); LRMS: m+1 (291.0,4.228 min.).

ethyl biohenyl-3-ylacetate (2-2)

To a mixture of (2-1) (5.0 g, 17.2 mmol), phenylboronic acid (51.7 mmol, 3 equiv), and tetrakis(triphenylphosphine)palladium (6.9 mmol, 0.4 equiv), were added toluene (100 mL), 2M solution of Na₂CO₃ (20 mL), and ethanol (20 mL). The reaction mixture was purged and refluxed at 90° C. for 18 h. The mixture was filtered through a celite pad and concentrated. Purification by flash chromatography (EtOAc/Hex) afforded brown oil as product. ¹H NMR (600 MHz, CDCl₃): δ=7.58 (dd, 2 H), 7.50 (m, 2 H), 7.43 (t, 2 H), 7.39 (t, 1 H), 7.34 (t, 1 H), 7.27 (dt, 1 H), 4.17 (q, 2 H), 3.68 (s, 2 H), 1.26 (t, 3 H). LRMS: m+1 (241.1, 2.885 min.).

biphenyl-3-ylacetic acid (2-3)

To a solution of (2-2) (2.25g, 9.4 mmol) in THF (48 mL) was added 4N solution of LIOH (12 mL). The mixture was stirred capped at room temperature for 16 h. The pH of the mixture was adjusted to 3 by addition of 1N HCl, followed by extraction with EtOAc (×5). The combined EtOAc extracts were dried (MgSO₄) and the solvent was removed. Purification by flash chromatography (EtOAc/Hex) gave white solid as product. ¹H NMR (600 MHz, CDCl₃): δ=10.96 (s, 1 H), 7.57 (dd, 2 H), 7.50 (m, 2 H), 7.42 (m, 3 H), 7.34 (t, 1 H), 7.27 (dt, 1 H), 3.68 (s, 2 H); LRMS: m+1 (213.1, 2.304 min.).

methyl 2-[(biphenyl-3-,ylacetyl)amino]-4-chloro-5-cyanobenzoate (2-4)

To a solution of (2-3) (1.12 g, 5.3 mmol) in CH₂Cl₂ (75 mL) was added oxalyl chloride (10.6 mmol, 2 equiv), followed by a drop of DMF. The mixture was stirred at room temperature for 1 h. Upon removal of the solvent, amine (1.12 g, 5.3 nmmol) and 1,2-dichloroethane were added and the mixture was refluxed at 100° C. for 18 h. Once the reaction mixture was brought to the room temperature, MeOH was added to quench the reaction mixture and then the solvent was removed. Upon purification by flash chromatography (EtOAc/Hex), the desired product was obtained as white solid. ¹H NMR (600 MHz, CDCl₃): δ=11.33 (s, 1 H), 9.03 (s, 1 H), 8.28 (s, 1 H), 7.61 (dd, 2 H), 7.56 (m, 2 H), 7.44 (m, 3 H), 7.34 (m, 2 H), 3.86 (s, 3 H), 3.84 (s, 2 H); LRMS: m+1 (405.1, 3.150 min).

3-biphenyl-3-yl-7-chloro4-hydroxy-2-oxo-1,2-dihydroquinoline-6-arbonitrile (2-5)

To a solution of (2-4) (2.03 g, 5.0 mmol) in THF (50 mL) was added 1 M solution of sodium bis (TMS) amide in THF (11 mL). The mixture was stirred at room temperature for 1 h. Upon addition of 6 N HCl, white solid appeared. Filtration afforded white solid as product (1.46 g, 78%). ¹H NMR (600 MHz, (CD₃)₂SO): δ=11.96 (s, 1 H), 10.83 (s, 1 H), 8.37 (s, 1 H), 7.62 (d, 2 H), 7.59 (m, 2 H), 7.48 (t, 1 H), 7.43 (m, 3 H), 7.32 (m, 2 H). ¹³ C NMR (150.8 MHz, (CD₃)₂SO): δ=163.5, 157.0, 142.1, 140.9, 140.5, 136.5, 133.4, 131.4, 130.8, 130.0, 129.6, 129.2, 128.1, 127.3, 126.5, 116.9, 116.3, 115.7, 114.5, 104.7. LRMS: m+1 (373.0, 2.487 min.).

The compounds shown in Table 2 were synthesized according to the Reaction Scheme 2.

TABLE 2

| Cmp | Structure | Name | LRMS m/z (M + H) |
|---|---|---|---|
| 2-6 | 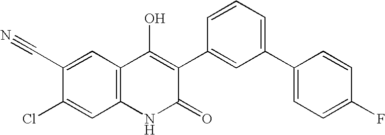 | 7-chloro-3 -(4′-fluorobiphenyl-3-yl)-4-hydroxy-2-oxo-1,2-dihydroquinoline-6-carbonitrile | LRMS m/z (M + H)391.1 found, 391.1required. |
| 2-7 | 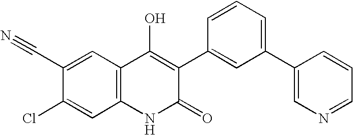 | 7-chloro-4-hydroxy-2-oxo-3-(3-pyridin-3-ylphenyl)-1,2-dihydroquinoline-6-carbonitrile | LRMS m/z (M + H)374.1 found, 374.1required. |
| 2-8 | 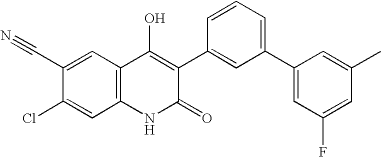 | 7-chloro-3-(3′,5′-difluorobiphenyl-3-yl)-4-hydroxy-2-oxo-1,2-dihydroquinoline-6-carbonitrile | LRMS m/z (M + H)409.1 found, 409.1required. |
| 2-9 | 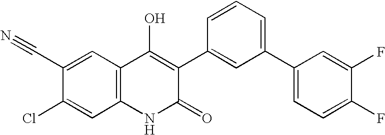 | 7-chloro-3-(3′,4′,-difluorobiphenyl-3-yl)-4-hydroxy-2-oxo-1,2-dihydroquinoline-6-carbonitrile | LRMS m/z (M + H)409.1 found, 409.1required. |
| 2-10 | 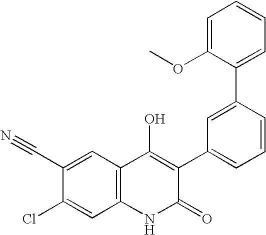 | -chloro-4-hydroxy-3-(2′-methoxybiphenyl-3-yl)-2-oxo-1,2-dihydroquinoline-6-carbonitrile | LRMS m/z (M + H)403.1 found, 403.1required. |
| 2-11 | 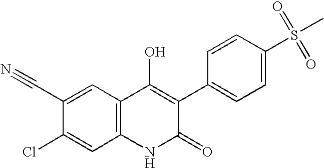 | 7-chloro-4-hydroxy-3-[4-(methylsulfonyl)phenyl]-2-oxo-1,2-dihydroquinoline-6-carbonitrile | LRMS m/z (M + H)375.1 found, 375.1required. |
| 2-12 | 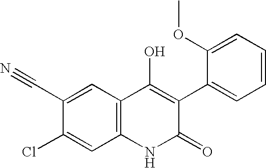 | 7-chloro-4-hydroxy-3-(2-methoxyphenyl)-2-oxo-1,2-dihydroquinoline-6-carbonitrile | LRMS m/z (M + H)327.1 found, 327.1required. |
| 2-13 | 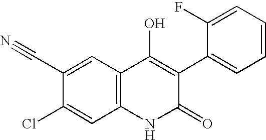 | 7-chloro-3-(2-fluorophenyl)-4-hydroxy-2-oxo-1,2-dihydroquinoline-6-carbonitrile | LRMS m/z (M + H)315.0 found, 315.0required. |
| 2-14 | 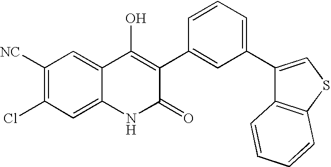 | 3-[3-(1-benzothien-3-yl)phenyl[-7-chloro-4-hydroxy-2-oxo-1,2-dihydroquinoline-6-carbonitrile | LRMS m/z (M + H)429.0 found, 429.0required. |
| 2-15 | 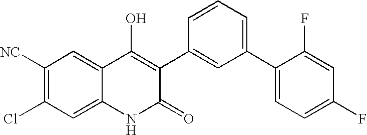 | 7-chloro-3-(2′,4′-difluorobiphenyl-3-yl)-4-hydroxy-2-oxo-1,2-dihydroquinoline-6-carbonitrile | LRMS m/z (M + H)409.0 found, 409.0required. |
| 2-16 | 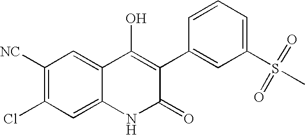 | 7-chloro-4-hydroxy-3-[3-(methylsu1fony1)phenyl]2-oxo-1,2-dihydroquinoline-6-carbonitrile | LRMS m/z (M + H)375.0 found, 375.0required. |
| 2-17 | 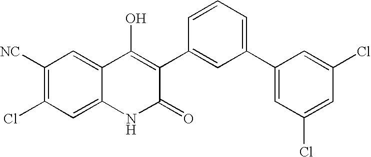 | 7-chloro-3-(3′,5′-dichlorobiphenyl-3-yl)4-hydroxy-2-oxo-1,2-dihydroquinoline-6-carbonitrile | LRMS m/z (M + H)441.0 found, 441.0required. |
| 2-18 | 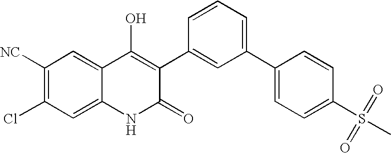 | 7-chloro-4-hydroxy-3-[4′-(methylsulfonyl)biphenyl-3-yl]-2-oxo-1,2-dihydroquinoline-6-carbonitrile | LRMS m/z (M + H)451.0 found, 451.0required. |
| 2-19 | 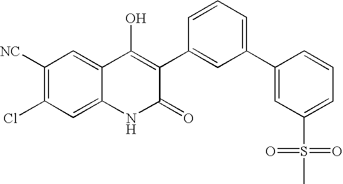 | 7-chloro-4-hydroxy-3-[3′-(methylsulfonyl)biphenyl-3-yl]-2-oxo-1,2-dihydroquinoline-6-carbonitrile | LRMS m/z (M + H)451.0 found, 451.0required. |
| 2-20 | 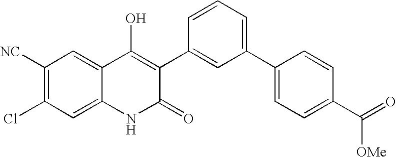 | methyl3′-(7-chloro-6-cyano-4-hydroxy-2-oxo-1,2-dihydroquinolin-3-yl)biphenyl-4-carboxylate | LRMS m/z (M + H)431.1 found, 431.1required. |
| 2-21 | 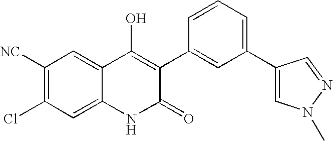 | 7-chloro-4-hydroxy-3-[3-(1-methyl-1H-pyrazol-4-yl)phenyl]-2-oxo-1,2-dihydroquinoline-6-carbonitrile | LRMS m/z (M + H)377.1 found, 377.1required. |
| 2-22 | 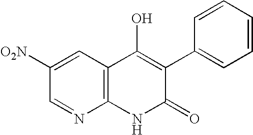 | 4-hydroxy-6-nitro-3-phenyl-1,8-naphthyridin-2(1H)-one | LRMS m/z (M + H)284.1 found, 284.1required. |
| 2-23 | 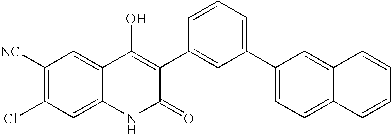 | 7-chloro-4-hydroxy-3-[3-(2-naphthyl)pheny1]-2-oxo-1,2-dihydroquino1ine-6-carbonitrile | LRMS m/z (M + H)423.1 found, 423.1required. |
| 2-24 | 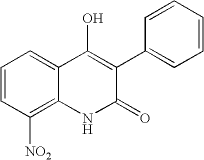 | 4-hydroxy-8-nitro-3-phenylquinolin-2(1H)-one | LRMS m/z (M + H)283.1 found, 283.1required. |
| 2-25 | 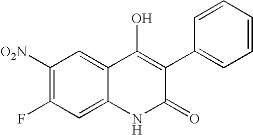 | 7-fluoro-4-hydroxy-6-nitro-3-phenylquino1in-2(1H)-one | LRMS m/z (M + H)300.1 found, 300.1required. |
| 2-26 | 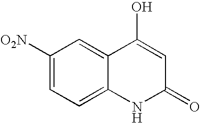 | 4-hydroxy-6-nitroquinolin-2(1H)-one | LRMS m/z (M + H)207.1 found, 207.1required. |
| 2-27 | 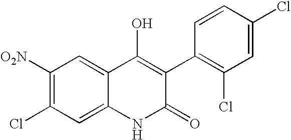 | 7-chloro-3-(2,4-dichlorophenyl)-4-hydroxy-6-nitroquinolin-2(1H)-one | LRMS m/z (M + H)384.9 found, 384.9required. |
| 2-28 | 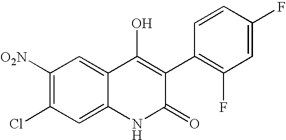 | 7-chloro-3-(2,4-difluorophenyl)-4-hydroxy-6-nitroquinolin-2(1H)-one | LRMS m/z (M + H)353.0 found, 353.0required. |
| 2-29 | 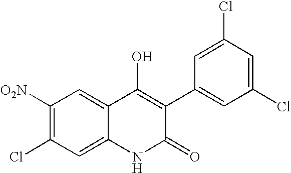 | 7-chloro-3-(3,5-dichlorophenyl)-4-hydroxy-6-nitroquino1in-2(1H)-one | LRMS m/z (M + H)384.9 found, 384.9required. |
| 2-30 | 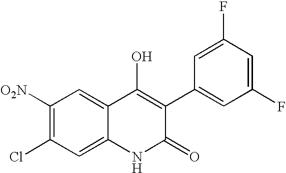 | 7-chloro-3-(3,5-difluorophenyl)-4-hydroxy-6-nitroquinolin-2(1H)-one | LRMS m/z (M + H)353.0 found, 353.0required. |
| 2-31 | 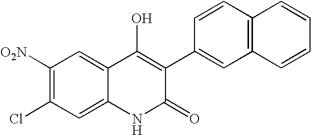 | 7-chloro-4-hydroxy-3-(2-naphthyl)-6-nitroquinolin-2(1H)-one | LRMS m/z (M + H)367.0 found, 367.0required. |
| 2-32 | 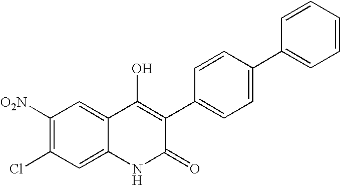 | 3-biphenyl-4-yl-7-chloro-4-hydroxy-6-nitroquinolin-2(1H)-one | LRMS m/z (M + H)393.0 found, 393.0required. |
| 2-33 | 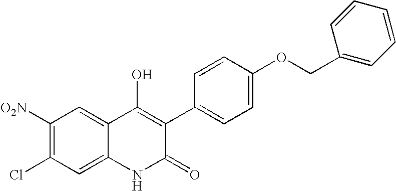 | 3-[4-(benzyloxy)phenyl]-7-chloro-4-hydroxy-6-nitroquinolin-2(1H)-one | LRMS m/z (M + H)423.1 found, 423.1required. |
| 2-34 | 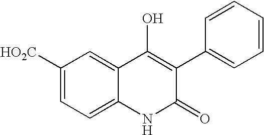 | 4-hydroxy-2-oxo-3-phenyl-1,2-dihydroquinoline-6-carboxylic acid | LRMS m/z (M + H)282.1 found, 282.1required. |
| 2-35 | 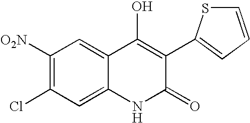 | 7-chloro-4-hydroxy-6-nitro-3-(2-thienyl)quinolin-2(1H)-one | LRMS m/z (M + H)323.0 found, 323.0required. |
| 2-36 | 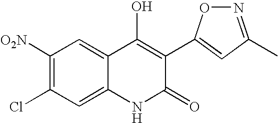 | 7-chloro-4-hydroxy-3-(3-methylisoxazol-5-yl)-6-nitroquinolin-2(1H)-one | LRMS m/z (M + H)322.0 found, 322.0required. |
| 2-37 | 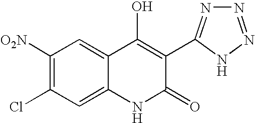 | 7-chloro-4-hydroxy-6-nitro-3-(1H-tetrazol-5-yl)quinolin-2(1H)-one | LRMS m/z (M + H)309.0 found, 309.0required. |
| 2-38 | 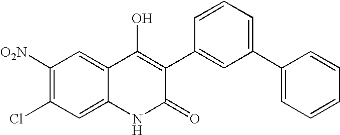 | 3-biphenyl-3-yl-7-chloro-4-hydroxy-6-nitroquinolin-2(1H)-one | LRMS m/z (M + H)393.1 found, 393.1required. |

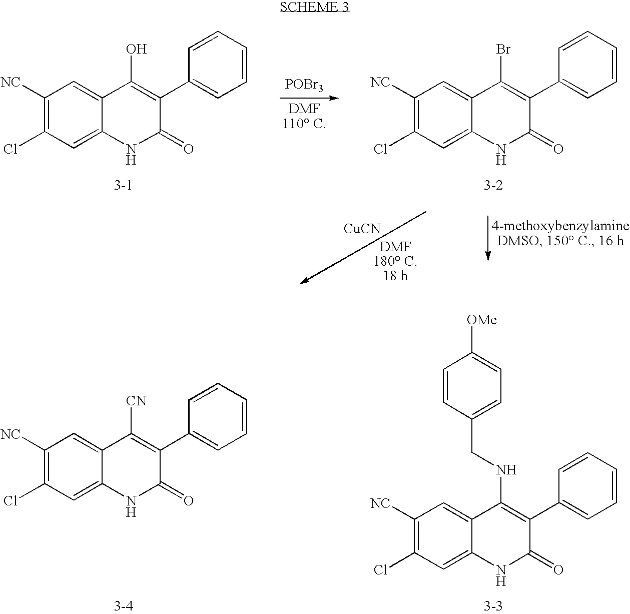

7-chloro4-[(4-methoxybenzyl)amino]-2-oxo-3-phenyl-1,2-dihydroquinoline-6-carbonitrile (3-3) and 7-chloro-2-oxo-3-phenyl-1,2-dihydroquinoline4,6-dicarbonitrile (3-4)

7-chloro4-hvdroxy-2-oxo-3-phenyl-1,2-dihydroquinoline-6-carbonitrile (3-1)

To a solution of 4 (1.0 g, 3.37 mmol) in DMF (4 mL) was added POBr₃ (966 mg, 1.0 equiv). The mixture was refluxed at 110° C. for 15 h, cooled to r. t., and then diluted with H₂O (4 mL). The pH of the mixture was adjusted to 7 by addition of 2 M Na₂CO₃, followed by extraction with CH₂Cl₂ (×5). The combined CH₂Cl₂ extracts were dried (MgSO₄). Removal of the solvent provided the desired compound as a cream colored solid (230 mg, 19%). ¹H NMR (600 MHz, (CD₃)₂SO): δ=12.71 (s, 1 H), 8.39 (s, 1 H), 7.58 (s, 1 H), 7.44 (t, 2 H), 7.40 (d, 1 H), 7.29 (d, 2 H). LRMS: [M+1]⁺: 358.9 (1.390 min).

4-bromo-7-chloro-2-oxo-3-phenyl-1,2-dihydroquinoline-6-carbonitrile (3-2)

To a solution of 1 (200 mg, 0.56 mmol) in DMSO (0.5 mL) was added 4-methoxybenzylamine (72 μl, 1.0 equiv). The mixture was stirred at 150° C. for 16 h, cooled to r. t., and then diluted with CH₂Cl₂ (4 mL). Purification by flash chromatography (EtOAc-hexanes) afforded cream colored solid as the desired product (40 mg, 17%). ¹H NMR (600 MHz, CDCl₃): δ=11.64 (s, 1 H), 8.01 (s, 1 H), 7.43 (t, 2 H), 7.38 (t, 1 H), 7.34 (s, 1 H), 7.27 (d, 3 H), 6.98 (d, 2 H), 6.82 (d, 2 H), 4.08 (s, 2 H), 3.79 (s, 3 H). LRMS: [M+1]⁺: 416.1 (1.806 min).

7-chloro4-[(4-methoxybenzyl)amino]-2-oxo-3-phenyl-1,2-dihydroquinoline-6-carbonitrile (3-3)

A solution of 2 (40 mg, 0.10 mmol) in trifluoroacetic acid (2 mL) was stirred at 65° C. for 1 h. Acid was removed under reduced pressure. The mixture was diluted with ethyl acetate, and washed with 10% NaOH solution. The organic layer was dried (MgSO₄), and concentrated. Purification by reversed-phase HPLC followed by a NaHCO₃ wash gave cream colored solid as the desired product (7 mg, 24%). ¹H NMR (600 MHz, (CD₃)₂SO): δ=12.01 (s, 1 H), 7.89 (s, 1 H), 7.40 (t, 3 H), 7.24 (d, 2 H), 6.68 (s, 2 H), 6.57(s, 1 H). LRMS: [M+1]⁺: 296.1 (1.308 min). 7-chloro-2-oxo-3-phenyl-1,2-dihydroquinoline4,6-dicarbonitrile (3-4)

To a solution of 1 (189 mg, 0.53 mmol) in DMF (3 mL) was added CuCN (2.1 mmol, 4.0 equiv). The mixture was stirred at 180° C. for 18 h, cooled to r. t., and then diluted with MeOH (4 mL). Purification by reversed-phase HPLC followed by a NaHCO₃ wash gave yellow solid as the desired product (65 mg, 40%). ¹H NMR (600 MHz, (CD₃)₂SO): δ=12.92 (s, 1 H), 8.28 (s, 1 H), 7.57 (m, 3 H), 7.52 (m, 3 H). ¹³C N (150 MHz, (CD₃)₂SO): δ=160.182, 143.072, 142.193, 137.571, 133.221, 130.551, 128.800, 118.902, 117.008, 116.173, 114.705, 106.608.LRMS: [M+1]⁺: 306.0(1.238 min).

The compounds shown in Table 3 were synthesized according to the Reaction Scheme 3.

TABLE 3

| Cmp | Structure | Name | LRMS m/z (M + H) |
|---|---|---|---|
| 3-5 | 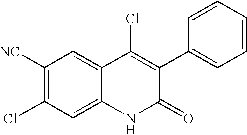 | 4,7-dichloro-2-oxo-3-phenyl-1,2-dihydroquinoline-6-carbonitrile | LRMS m/z (M + H)315.0 found, 315.0required. |
| 3-6 | 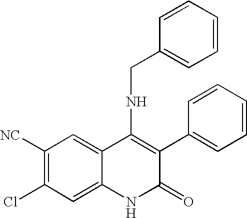 | 4-(benzylamino)-7-chloro-2-oxo-3-phenyl-1,2-dihydroquinoline-6-carbonitrile | LRMS m/z (M + H)386.1 found, 386.1required. |
| 3-7 | 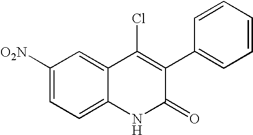 | 4-chloro-6-nitro-3-phenylquinolin-2(1H)-one | LRMS m/z (M + H)301.0 found, 301.0required. |

### Example 1

Example 1 is provided below to further illustrate different features and advantages of the present invention.

### Example 1

### Inhibition of Fatty Acid Synthase Enzyme

Fatty acid synthase (FAS) catalyzes the following reaction: acetyl-CoA+n malonyl-CoA+2n NADPH+2n H+=long-chain fatty acid+(n+1) coenzyme A [CoA]+n CO₂+2n NADP+. FAS activity is commonly determined by measuring (1) the generation of long-chain fatty acid [by radioactive tracer], (2) the loss of NADPH [by inherent NADPH aborbance or fluorescence] or (3) the generation of free CoA [by reaction with a sulfhydryl-reactive dye]. Here, FAS enzymatic activity in vitro was determined by measuring FAS-generated CoA with the sulfhydryl-reactive dye 7-Diethylamino-3-(4-maleimidophenyl)4-methylcoumarin [CPM]. Unreacted CPM is nonfluorescent. However, the CPM-CoA adduct exhibits fluorescence with a peak excitation wavelength of 380 nm and a peak emission wavelength of 475 nm. An assay for FAS activity using CPM was performed as follows: “FAS Buffer”, “FAS Enzyme”, “FAS Substrate”, and “CPM Stop Buffer” were made fresh from the components listed in the table below. Compounds were serially diluted 1:3 in anhydrous DMSO just prior to start of assay. 9 μL of FAS enzyrme was added to each well of a black Corning low volume, 384-well, non-binding surface microplate using a Matrix PlateMate Plus automated pipettor with a 384/30 μL head and Matrix extended-length 30 μL tips. 0.5 μL from each serially-diluted compound plate was then added to enzyme a using Matrix PlateMate Plus. Plates were incubated for 20 minutes at ambient temperature. 1 μL FAS Substrate was added to each well using Matrix PlateMate Plus. Plate was incubated 11 minutes at ambient temperature. The FAS reaction was stopped by the addition of 2.2 μL CPM Stop Buffer to each well using Matrix PlateMate Plus. Plate was incubated 15 minutes at ambient temperature. A Perldn-Elmer ViewLux uHTS microplate imager was then used to measure CPM fluorescence in each well of the plate (ViewLux reading used 380 nm excitation filter, 475 nm emission filter and D400 mirror). The percentage reduction of signal in wells with compound was then used to determine potency of each compound by a non-linear fit of percent inhibition vs. dose.

| | Total | |
|---|---|---|
| Buffer Name | Volume | Buffer Components |
| FAS Buffer | 50 ml | 49.5 ml 75 mM potassium phosphate buffer, |
| | | pH 6.8 [75 mM] |
| | | 50 μl 50 mM EDTA [50 μM] |
| | | 50 μl 1M ascorbate [1 mM] |
| | | 50 μl 25 mM acetyl-CoA [25 μM] |
| | | 500 μl 15 mM NADPH [150 μM] |
| FAS Enzyme | 30 mL | 30 mL FAS Buffer |
| | | 137 μL FAS Enzyme* @ 441 μU/μL [2 μU/μL |
| | | in rxn] |
| CPM Stop | 25 ml | 10 ml absolute ethanol [40%] |
| Buffer | | 15 ml 75 mM pottasium phosphate, pH 6.8 [60%] |
| | | 50 μl 66 mM CPM [132 μM] |
| FAS | 20 ml | 20 ml FAS Buffer |
| Substrate | | 168 μL 25 mM malonyl-CoA [210 μM] |

*human enzyme isolated from SK-BR3 cells

Specific compounds of the instant invention were tested in the assay described above and were found to have IC₅₀ of ≦50 μM against substrate.

1. A compound of the Formula A: 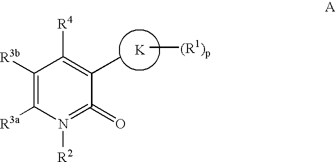
wherein:
a is 0 or 1; b is 0 or 1; m is 0, 1, or 2; p is 0, 1, 2, 3, 4 or 5;
Ring K is: aryl, C₃-C₈cycloalkyl or heterocyclyl;
R¹ is selected from: (C═O)ₐO_{b}C₁-C₁₀ alkyl, (C═O)ₐO_{b} aryl, (C═O)ₐO_{b}C₂-C₁₀ alkenyl, (C═O)ₐO_{b}C₂-C₁₀ alkynyl, CO₂H, halo, OH, O_{b}C₁-C₆ perfluoroalkyl, (C═O)ₐNR⁷R⁸, CN, (C═O)ₐO_{b}C₃-C₈ cycloalkyl, S(O)ₘNR⁷R⁸, S(O)ₘ—(C₁-C₁₀)alkyl, and (C═O)ₐO_{b}heterocyclyl, said alkyl, aryl, alkenyl, alkynyl, cycloalkyl, and heterocyclyl is optionally substituted with one or more substituents selected from R⁶;
R² is selected from: H, (C═O)ₐO_{b}C₁-C₁₀ alkyl, (C═O)ₐO_{b} aryl, (C═O)ₐO_{b}C₂-C₁₀ alkenyl, (C═O)ₐO_{b}C₂-C₁₀ alkynyl, CO₂H, halo, OH, O_{b}C₁-C₆ perfluoroalkyl, (C═O)ₐNR⁷R⁸, CN, (C═O)ₐO_{b}C₃-C₈ cycloalkyl, S(O)ₘNR⁷R⁸, S(O)ₘ—(C₁-C₁₀)alkyl, and (C═O)aObheterocyclyl, said alkyl, aryl, alkenyl, alkynyl, cycloalkyl, and heterocyclyl is optionally substituted with one or more substituents selected from R⁶;
R^{3a} and R^{3b} are independently selected from: H, (C═O)ₐO_{b}C₁-C₁₀ alkyl, (C═O)ₐO_{b} aryl, (C═O)ₐO_{b}C₂-C₁₀ alkenyl, (C═O)ₐO_{b}C₂-C₁₀ alkynyl, CO₂H, halo, OH, O_{b}C₁-C₆ perfluoroalkyl, (C═O)ₐNR⁷R⁸, CN, (C═O)ₐO_{b}C₃-C₈ cycloalkyl, S(O)ₘNR⁷R⁸, S(O)ₘ—(C₁-C₁₀)alkyl, SH, NO₂, and (C═O)ₐO_{b}heterocyclyl, said alkyl, aryl, alkenyl, alkynyl, cycloalkyl, and heterocyclyl is optionally substituted with one or more substituents selected from R⁶, or R^{3a} and R^{3b} can be combined to form a fused aryl or heterocycle which are optionally substituted with one or more substituents selected from R⁶;
R⁴ is selected from: H, (C═O)ₐO_{b}C₁-C₁₀ alkyl, (C═O)ₐO_{b} aryl, (C═O)ₐO_{b}C₂-C₁₀ alkenyl, (C═O)ₐO_{b}C₂-C₁₀ alkynyl, CO₂H, halo, OH, O_{b}C₁-C₆ perfluoroalkyl, (C═O)ₐNR⁷R⁸, CN, (C═O)ₐO_{b}C₃-C₈ cycloalkyl, S(O)ₘNR⁷R⁸, S(O)ₘ—(C₁-C₁₀)alkyl, and (C═O)ₐO_{b}heterocyclyl, said alkyl, aryl, alkenyl, alkynyl, cycloalkyl, and heterocyclyl is optionally substituted with one or more substituents selected from R⁶;
R⁶ is: (C═O)ₐO_{b}C₁-C₁₀ alkyl, (C═O)ₐO_{b}aryl, C₂-C₁₀ alkenyl, C₂-C₁₀ alkynyl, (C═O)ₐO_{b}heterocyclyl, CO₂H, halo, CN, OH, O_{b}C₁-C₆ perfluoroalkyl, Oₐ(C═O)_{b}NR⁷R⁸, oxo, CHO, (N═O)R⁷R⁸, S(O)ₘNR⁷R⁸, S(O)ₘ—(C₁-C₁₀)alkyl, SH, SO₂—CF₃, NO₂, or (C═O)ₐO_{b}C₃-C₈ cycloalkyl, said alkyl, aryl, alkenyl, alkynyl, heterocyclyl, and cycloalkyl optionally substituted with one or more substituents selected from R^{6a}, and two R⁶ substituents can be combined to form a fused aryl or heterocycle which are optionally substituted with one or more substituents selected from R^{6a};
R^{6a} is selected from: (C═O)ₐO_{b}(C₁-C₁₀)alkyl, Oₐ(C₁-C₃)perfluoroalkyl, (C₀-C₆)alkylene-S(O)ₘR^{a}, oxo, OH, halo, CN, (C₂-C₁₀)alkenyl, (C₂-C₁₀)alkynyl, (C₃-C₆)cycloalkyl, (C₀-C₆)alkylene-aryl, (C₀-C₆)alkylene-heterocyclyl, (C₀-C₆)alkylene-N(R^{b})₂, C(O)R^{a}, (C₀-C₆)alkylene-CO₂R^{a}, C(O)H, and (C₀-C₆)alkylene-CO₂H, said alkyl, alkenyl, alkynyl, cycloalkyl, aryl, and heterocyclyl is optionally substituted with up to three substituents selected from R^{b}, OH, (C₁-C₆)alkoxy, halogen, CO₂H, CN, O(C═O)C₁-C₆ alkyl, oxo, and N(R^{b})₂;
R⁷ and R⁸ are independently selected from: H, (C═O)O_{b}C₁-C₁₀ alkyl, (C═O)O_{b}C₃-C₈ cycloalkyl, (C═O)O_{b}aryl, (C═O)O_{b}heterocyclyl, C₁-C₁₀ alkyl, aryl, C₂-C₁₀ alkenyl, C₂-C₁₀ alkynyl, heterocyclyl, C₃-C₈ cycloalkyl, S(O)ₘR^{a}, and (C═O)NR^{b}₂, said alkyl, cycloalkyl, aryl, heterocylyl, alkenyl, and alkynyl is optionally substituted with one or more substituents selected from R^{6a}, or R⁷ and R⁸ can be taken together with the nitrogen to which they are attached to form a monocyclic or bicyclic heterocycle with 3-7 members in each ring and optionally containing, in addition to the nitrogen, one or two additional heteroatoms selected from N, O and S, said monocylcic or bicyclic heterocycle optionally substituted with one or more substituents selected from R^{6a};
R^{a} is H, (C₁-C₆)alkyl, (C₃-C₆)cycloalkyl, aryl, or heterocyclyl; and
R^{b} is independently H, (C₁-C₆)alkyl, aryl, heterocyclyl, (C₃-C₆)cycloalkyl, (C═O)OC₁-C₆ alkyl, (C═O)C₁-C₆ alkyl or S(O)ₘR^{a};
or a pharmaceutically acceptable salt or a stereoisomer thereof.

2. The compound according to claim 1 of the Formula B: 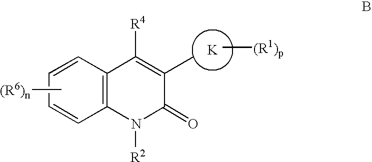
wherein:
n is 0, 1, 2, 3 or 4;
Ring K is selected from: phenyl, 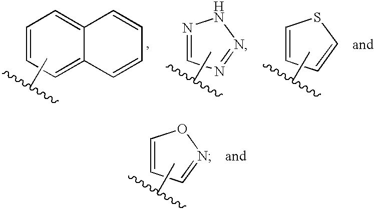
all other substituents and variables are as defined in claim 1;
or a pharmaceutically acceptable salt or a stereoisomer thereof.

3. The compound according to claim 2 of the Formula C: 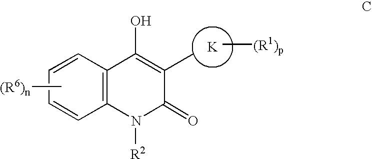
wherein:
all other substituents and variables are as defined in claim 2;
or a pharmaceutically acceptable salt or a stereoisomer thereof.

4. The compound according to claim 3 of the Formula D: 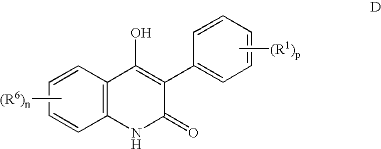
wherein:
all other substituents and variables are as defined in claim 2;
or a pharmaceutically acceptable salt or a stereoisomer thereof.

5. The compound according to claim 4 of the Formula E: 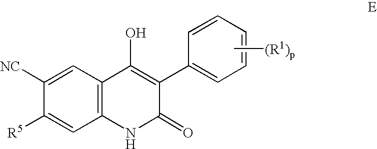
wherein:
R⁵ is selected from: H, halo and (C₁-C₆)alkyl; and
all other substituents and variables are as defined in claim 1;
or a pharmaceutically acceptable salt or a stereoisomer thereof.

6. A compound according to claim 1 which is selected from:
7-chloro-4-hydroxy-2-oxo-3-phenyl-1,2-dihydroquinoline-6-carbonitrile;
3-biphenyl-3-yl-7-chloro-4-hydroxy-2-oxo-1,2-dihydroquinoline-6-carbonitrile;
7-chloro-3-(4′-fluorobiphenyl-3-yl)-4-hydroxy-2-oxo-1,2-dihydroquinoline-6-carbonitrile;
7-chloro-4-hydroxy-2-oxo-3-(3-pyridin-3-ylphenyl)-1,2-dihydroquinoline-6-carbonitrile;
7-chloro-3-(3′,5′-difluorobiphenyl-3-yl)-4-hydroxy-2-oxo-1,2-dihydroquinoline-6-carbonitrile;
7-chloro-3-(3′,4′-difluorobiphenyl-3-yl)-4-hydroxy-2-oxo-1,2-dihydroquinoline-6-carbonitrile;
-chloro-4-hydroxy-3-(2′-methoxybiphenyl-3-yl)-2-oxo-1,2-dihydroquinoline-6-carbonitrile;
7-chloro-4-hydroxy-3-[4-(methylsulfonyl)phenyl]-2-oxo-1,2-dihydroquinoline-6-carbonitrile;
7-chloro-4-hydroxy-3-(2-methoxyphenyl)-2-oxo-1,2-dihydroquinoline-6-carbonitrile;
7-chloro-3-(2-fluorophenyl)-4-hydroxy-2-oxo-1,2-dihydroquinoline-6-carbonitrile;
3-[3-(1-benzothien-3-yl)phenyl]-7-chloro-4-hydroxy-2-oxo-1,2-dihydroquinoline-6-carbonitrile;
7-chloro-3-(2′,4′-difluorobiphenyl-3-yl)-4-hydroxy-2-oxo-1,2-dihydroquinoline-6-carbonitrile;
7-chloro-4-hydroxy-3-[3-(methylsulfonyl)phenyl]-2-oxo-1,2-dihydroquinoline-6-carbonitrile;
7-chloro-3-(3′,5′-dichlorobiphenyl-3-yl)-4-hydroxy-2-oxo-1,2-dihydroquinoline-6-carbonitrile;
7-chloro-4-hydroxy-3-[4′-(methylsulfonyl)biphenyl-3-yl]-2-oxo-1,2-dihydroquinoline-6-carbonitrile;
7-chloro-4-hydroxy-3-[3′-(methylsulfonyl)biphenyl-3-yl]-2-oxo-1 ,2-dihydroquinoline-6-carbonitrile;
methyl3′-(7-chloro-6-cyano-4-hydroxy-2-oxo-1,2-dihydroquinolin-3-yl)biphenyl-4-carboxylate;
7-chloro-4-hydroxy-3-[3-(1-methyl-1H-pyrazol-4-yl)phenyl]-2-oxo-1 ,2-dihydroquinoline-6-carbonitrile; and
7-chloro-4-hydroxy-3-[3-(2-naphthyl)phenyl]-2-oxo-1,2-dihydroquinoline-6-carbonitrile;
or a pharmaceutically acceptable salt or a stereoisomer thereof.

7. A pharmaceutical composition comprising a pharmaceutical carrier, and dispersed therein, a therapeutically effective amount of a compound of claim 1.

8. (canceled)

9. A method for treating cancer which comprises administering to a mammal in need thereof a therapeutically effective amount of a compound of claim 1.
